(19) United States
(12) Patent Application Publication
(10) Pub. No.: US 2019/0374479 A1
(43) Pub. Date: Dec. 12, 2019

(54) PARTICLES FOR DELIVERY OF BIOMOLECULES

(71) Applicants: The Brigham and Women's Hospital, Inc., Boston, MA (US); The Regents of the University of Michigan, Ann Arbor, MI (US)

(72) Inventors: Omid C. Farokhzad, Waban, MA (US); James Moon, Ann Arbor, MI (US); Sejin Son, Ann Arbor, MI (US)

(21) Appl. No.: 16/439,694

(22) Filed: Jun. 12, 2019

Related U.S. Application Data
(60) Provisional application No. 62/683,699, filed on Jun. 12, 2018.

(51) Int. Cl. A61K 9/51; A61P 37/04; A61K 39/39; A61P 35/00; A61K 39/00; B01J 13/04; B01J 13/20

(52) U.S. Cl. CPC A61K 9/5192; A61K 9/5146; A61K 9/5161; A61P 37/04; B01J 13/203; A61P 35/00; A61K 39/0011; B01J 13/04; A61K 39/39

(57) ABSTRACT

This disclosure relates to particles, compositions, methods of making, and methods of use thereof.

### CLAIM OF PRIORITY

This application claims priority under to U.S. Provisional Patent Application Ser. No. 62/683,699, filed on Jun. 12, 2018, the entire contents of which are hereby incorporated by reference.

### FEDERALLY SPONSORED RESEARCH OR DEVELOPMENT

This invention was made with Government support under Grant Nos. R01EB022563, R01AI127070, and R01CA210273 awarded by the National Institutes of Health, and Grant No. CAREER Award (1553831) awarded by National Science Foundation. The Government has certain rights in the invention.

### TECHNICAL FIELD

This disclosure relates to particles, compositions, methods of making, and methods of use thereof, and in particular to polysaccharide-coated particles comprising therapeutic biomolecules such as tumor antigen-encoding messenger RNA.

### BACKGROUND

Cancer is one of the leading causes of death in contemporary society. The number of new cancer cases and deaths is increasing each year. Currently, cancer incidence is 454.8 cases of cancer per 100,000 men and women per year, while cancer mortality is 71.2 cancer deaths per 100,000 men and women per year. Currently, there is no cure for this debilitating disease.

### SUMMARY

Despite great success of immunotherapy, it remains challenging to deliver adjuvant and antigen to specific immune cell subsets and elicit robust and durable immune responses. The recognition of pathogen-associated molecular patterns (PAMPs) by pathogen recognition receptors (PRRs) on antigen presenting cells (APCs) is the hallmark of innate host defense mechanism. This disclosure relates to nanocapsules containing polysaccharide-based PAMPs, for example, mannan- or dextran-based PAMPs. These nanocapsules exhibit strong adjuvanticity, promoting dramatic recruitment and activation of immune cells in lymph nodes (LNs) after in vivo administration. The nanocapsules are also useful as delivery vehicles for tumor antigen-encoding messenger RNA (mRNA) to the APCs. The nanocapsule protects RNA from enzymatic degradation, mediates its efficient uptake by APCs, improves translation of mRNA and production of the antigen protein, and promotes the production of the antibody by the lymphocyte. Notably, nanostructure of the capsule within the present claims, having the hollow structure, facilitates efficient lymph node drainage, significantly improving mRNA delivery to DCs in lymph nodes. In addition, the nanocapsule protects the mRNA from rapid enzymatic degradation and improves its pharmacokinetics.

In one general aspect, the present disclosure provides a method of making a particle, the method comprising:

i) combining a silica particle with a polyamine to obtain a polyamine-coated silica particle;

ii) combining the polyamine-coated silica particle with a crosslinker to obtain a crosslinked polyamine-coated silica particle;

iii) combining the crosslinked polyamine-coated silica particle with a polysaccharide to obtain a polysaccharide-coated silica particle; and

iv) combining the polysaccharide-coated silica particle with a fluoride source to remove the silica particle to obtain the particle.

In some embodiments, the silica particle is carboxylated.

In some embodiments, the polyamine is polyethyleneimine.

In some embodiments, the crosslinker comprises a —S—S— bridge.

In some embodiments, the crosslinker is 3,3′-dithiobispropionimidate having formula:

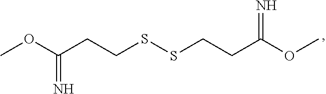

or a salt thereof.

In some embodiments, the polysaccharide comprises at least one aldehyde functional group.

In some embodiments, the polysaccharide comprises a pathogen-associated molecular pattern.

In some embodiments, the polysaccharide is selected from the group consisting of mannan and dextran.

In some embodiments, the fluoride source comprises ammonium fluoride.

In some embodiments:

the silica particle is carboxylated;

the polyamine is polyethyleneimine;

the crosslinker comprises a —S—S— bridge; and

the polysaccharide is selected from the group consisting of mannan and dextran, and the polysaccharide comprises at least one aldehyde functional group and a pathogen-associated molecular pattern.

In some embodiments, the present disclosure provides a particle comprising a shell comprising a crosslinked polyamine layer with a polysaccharide coating, prepared by any one of the methods described herein.

In some embodiments, the present disclosure provides a particle comprising: a shell comprising a crosslinked polyamine layer with a polysaccharide coating; wherein the shell surrounds a hollow core.

In some embodiments, the present disclosure provides a pharmaceutical composition comprising a particle as described herein.

In some embodiments, the present disclosure provides a method of modulating an immune response in a subject, the method comprising administering to the subject in need thereof a therapeutically effective amount of a particle as described herein.

In some embodiments, modulating the immune response comprises modulating a pattern recognition receptor (PRR) on the surface of an antigen-presenting cell (APC), promoting secretion of an inflammatory cytokine, and/or modulating a lymph node drainage.

In some embodiments, the method comprises administering the particle in combination with a vaccine.

In some embodiments, the present disclosure provides a method of treating a cancer, the method comprising administering to a subject in need thereof a therapeutically effective amount of a particle as described herein.

In some embodiments, the method comprises administering the particle in combination with an anti-cancer agent.

In some embodiments, the present disclosure provides a method of making a particle, the method comprising:

i) combining a silica particle with a polyamine to obtain a polyamine-coated silica particle;

ii) combining the polyamine-coated silica particle with a crosslinker to obtain a crosslinked polyamine-coated silica particle;

iii) combining the crosslinked polyamine-coated silica particle with a biomolecule to obtain a biomolecule-containing silica particle;

iv) combining the biomolecule-containing silica particle with a polysaccharide to obtain a polysaccharide-coated silica particle; and

iv) combining the polysaccharide-coated silica particle with a fluoride source to remove the silica particle to obtain the particle.

In some embodiments, the biomolecule is selected from the group consisting of a nucleic acid and a therapeutic protein.

In some embodiments, the nucleic acid is a messenger RNA (mRNA) encoding a cancer antigen.

In some embodiments, the therapeutic protein is a cancer antigen or an antibody useful in treating cancer.

In some embodiments:

the silica particle is carboxylated;

the polyamine is polyethyleneimine;

the crosslinker comprises a —S—S— bridge;

the polysaccharide is selected from the group consisting of mannan and dextran, and the polysaccharide comprises at least one aldehyde functional group and a pathogen-associated molecular pattern; and

the biomolecule is a messenger RNA (mRNA) encoding a cancer antigen.

In some embodiments, the present disclosure provides a particle comprising a shell comprising a crosslinked polyamine layer with a polysaccharide coating, and a biomolecule between the crosslinked polyamine layer and the polysaccharide coating, prepared by any one of the methods of the present disclosure.

In some embodiments, the present disclosure provides a particle comprising:

a shell comprising a crosslinked polyamine layer with a polysaccharide coating, and a biomolecule between the crosslinked layer the polysaccharide coating;

wherein the shell surrounds a hollow core.

In some embodiments, the present disclosure provides a pharmaceutical composition comprising a particle as described herein.

In some embodiments, the present disclosure provides a method of modulating an immune response in a subject, the method comprising administering to the subject in need thereof a therapeutically effective amount of a particle as described herein, wherein the biomolecule is useful in modulating an immune response in the subject.

In some embodiments, modulating the immune response comprises modulating a pattern recognition receptor (PRR) on the surface of an antigen-presenting cell (APC), promoting secretion of an inflammatory cytokine, modulating a lymph node drainage, and/or promoting expression of a disease-specific antigen by the antigen-presenting cell.

In some embodiments, the method comprises administering the particle in combination with an adjuvant.

In some embodiments, the present disclosure provides a method of treating a cancer, the method comprising administering to a subject in need thereof a therapeutically effective amount of a particle as described herein, wherein the biomolecule is useful in treating cancer.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 contains a scheme showing rational design and synthesis scheme of nanocapsule platform technology.

FIG. 2. a) Schematic illustration of the rational design and construction of mRNA-loaded nanocapsule (mRNA-nanocapsule). b) TEM images of nanocapsule before (top) and after (bottom) removal of a core silica nanoparticle. c) Composition of mRNA-nanocapsule in weight ratio. d) TEM images of Nanocapsule with multi-layered mRNA loading in high (top) and low (bottom) magnification. Evaluation of redox-responsive degradability of nanocapsule. e) TEM images of the degradation of nanocapsule and f) agarose gel image of mRNA released from nanocapsule in the presence or absence of DTT.

FIG. 3. a) Schematic illustration of the engagement and activation of DCs by Mann- or Dex-capsules via PAMP-PRR interaction, triggering strong immune responses by releasing inflammatory cytokines and upregulating co-stimulatory signals. b) Expression level of costimulatory signals of CD40 (top) and CD86 (bottom) c) secretion of pro-inflammatory cytokines of TNF-α (top) and IL-12 (bottom) from BMDCs pulsed with mOVA-Mann- or mOVA-Dex-capsule. d) Mechanistic evaluation of BMDCs engagement with nanocapsules via PAMP-PRR interaction in the presence of blocking antibodies against respective PRRs. IL-6 levels by Mann- (top) or Dex-capsule (bottom) e) Evaluation of mRNA translation efficacy by detection of EGFP, Confocal microscopy images of EGFP (green), Mann-capsule (pink) and nucleus (blue) in low (top) and high (middle) magnification and high resolution (bottom). f) Cytotoxicities with varying amounts of mOVA-nanocapsule. Error bars indicate s.e.m. Statistical significance was calculated by one-way ANOVA using the bonferroni post-test. P-value: ***, P<0.001; ****, P<0.0001.

FIG. 4. a) Schematic illustration of mRNA-loaded nanocapsule-fed BMDCs mediated antigen presentation and cytotoxic T-cell activation and expansion. b) Antigen presentation level and c) representative histograms of SIINFEKL peptide bound to H-2Kb of MHC class I. d) Proliferation rate, e) the number of, and f) representative histograms of OT-I T-cells in the presence of BMDCs that were engulfed with mRNA-loaded formulations. Error bars indicate s.e.m. Statistical significance was calculated by one-way ANOVA using the bonferroni post-test. P-value: ****, P<0.0001.

FIG. 5 a) An unique and well-defined hollow Sugar-capsule structure promotes lymph node drainage in vivo. The core-empty nanocapsule can diffuse into lymphatic vessel by deformation of their nanostructure, leading to enhanced accumulation in lymph nodes. b-d) fluorescence signal in the draining inguinal lymph nodes were quantified and imaged with IVIS at 3, 18, 36 and 72 hr post-injection. d) IVIS image and c) total radiant efficiency of excised inguinal lymph nodes at 3 hr post-injection. Mean fluorescence intensity (MFI) of local DCs in inguinal lymph nodes that are nanoparticle positive e) and costimulatory marker, CD86, positive (f).

FIG. 6. Anti-tumor efficacy of mOVA-loaded Mann- and Dex-nanocapsule. a) Schema of experiment design. C57BL/6 mice were inoculated with B16F100VA cells subcutaneously in the right flank on day 0 and immunized with all formulations with or without mRNA encoding ovalbumin on day 5, 10 and 15. b) Plot of average tumor growth curves, The data show mean±s.d., n=7, **P<0.01, and ****P<0.0001, analyzed by two-way ANOVA. Antigen-specific c) CD8 or d) CD4 T-cells measured by ELISPOT analysis among PBMCs on day 21. Tumor microenvironment analysis on day 23; e) Frequencies of CD8+ T cells among CD45+ cells, f) Frequencies and g) representative histograms of antigen-specific CD8+ T cells, h-k) Frequencies of CD4+ T cells, CD11c+ DCs among total cells, CD86+ cells among CD11c+ DCs and NK1.1+ natural killer cells among total cells. The data show mean±s.d., *P<0.05, **P<0.01, and ****P<0.0001, analyzed by one-away ANOVA, followed by Bonferroni multiple comparisons post-test. * in b indicates statistical differences compared to PBS on day 17; # in b indicates statistical differences compared to mOVA on day 17 and 19.

FIG. 7 Measurement of hydrodynamic size (a) and surface charge (b) as construction of polysaccharide-based nanocapsule.

FIG. 8 Colloidal stability of nanocapsule. Hydrodynamic size (a) and TEM images (b) of nanocapsule in the presence of high and low serum. Hydrodynamic size (c) and TEM image (d) of reconstituted frozen nanocapsule.

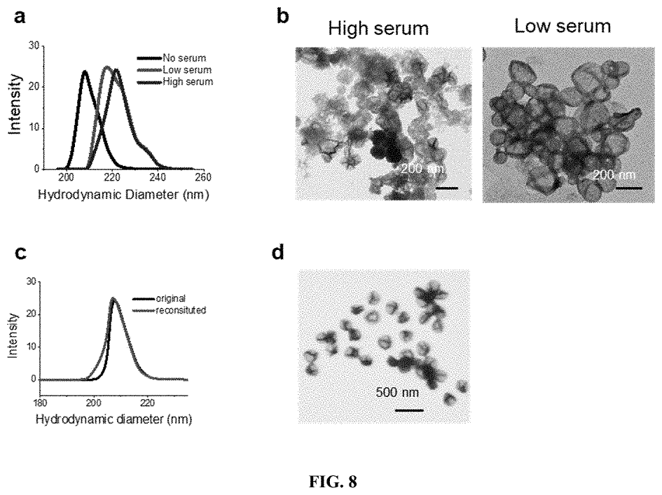

FIG. 9 Determination of mRNA loading in nanocapsule by GPC analysis. 100% of mRNA was loaded into PEI-coated siNP while no mRNA was loaded into bare-siNP in same condition.

FIG. 10 BMDC uptake of Dex- (top) and Mann-capsule (bottom) in 10% FBS supplemented RPMI media. Percent (%) (a) or mean fluorescence intensity (MFI) (b) of nanocapsule positive BMDCs, and time-dependent nanocapsule uptake (c).

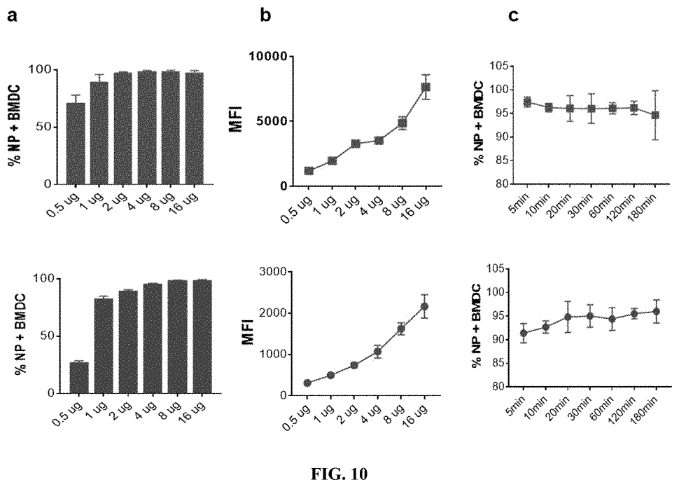

FIG. 11 Optimization of mRNA loading condition into nanocapsule by evaluation of EGFP mRNA translation efficiency in BMDCs in 10% FBS supplemented RPMI media. Single mRNA-layered Dex- or Mann-capsule shows dose-dependent mRNA translation efficacy and Dex-capsule is more efficacious than Mann-capsule. Multilayered mRNA-coating by layer-by-layer technique with PEI did not induce EGFP mRNA translation, indicating inefficient mRNA release from nanocapsule.

FIG. 12 Comparison of mRNA translation efficacy with conventional gene transfection agents, Lipofectamin and PEI in varying mRNA dose over incubation time. Fold change in MFI of EGFP fluorescence (a) and EGFP positive population in percent (b) of BMDCs compared with those of PBS treated. Representative histogram of EGFP-mRNA translation with all formulations (c).

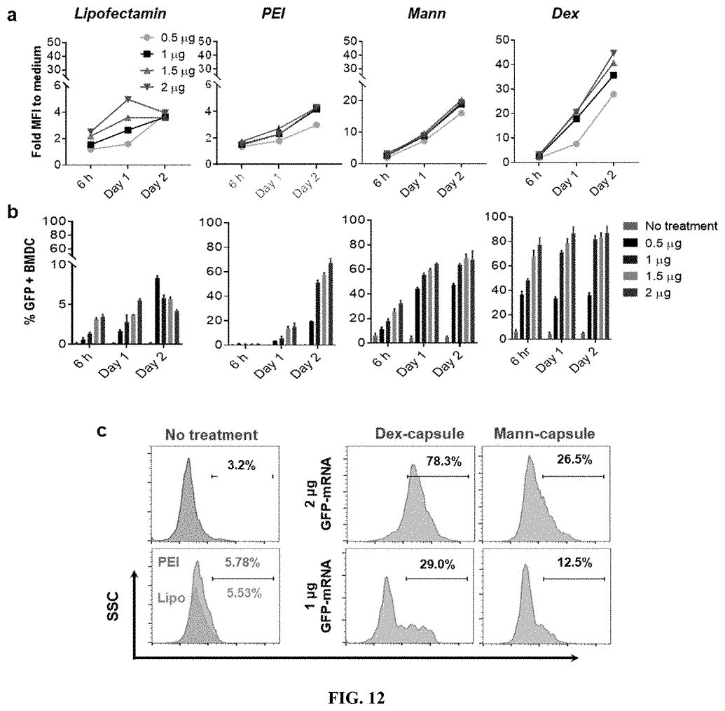

FIG. 13 Pathogen recognition receptor (PRR)-mediated Mann-capsule uptake (a) and EGFP-mRNA translation (b) efficiency in BMDCs in the absence and presence 10% mouse serum condition.

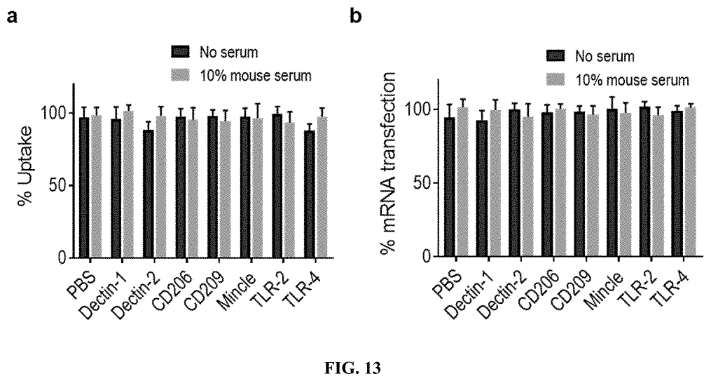

FIG. 14 Evaluation of dose- and time-dependent BMDCs activation by nanocapsule by the measurement of co-stimulatory markers and cytokine release. Percentage of positive of (a) and fold increase in MFI compared with PBS (b) of costimulatory markers and cytokine levels (c) in BMDCs.

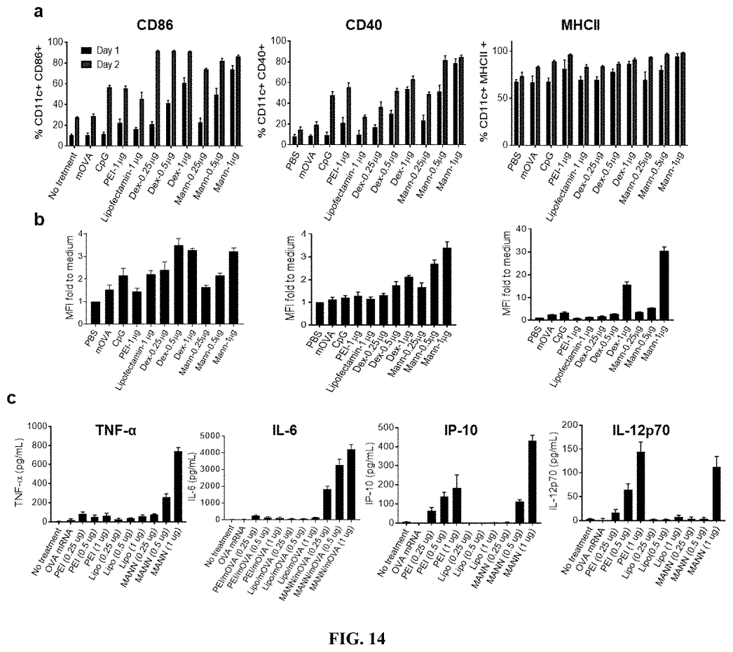

FIG. 15 Proliferation rate (a), the number (b), and c) representative histograms of OT-I T-cells in the presence of BMDCs that were treated with mOVA-loaded formulations for 4 hr or 25 hr.

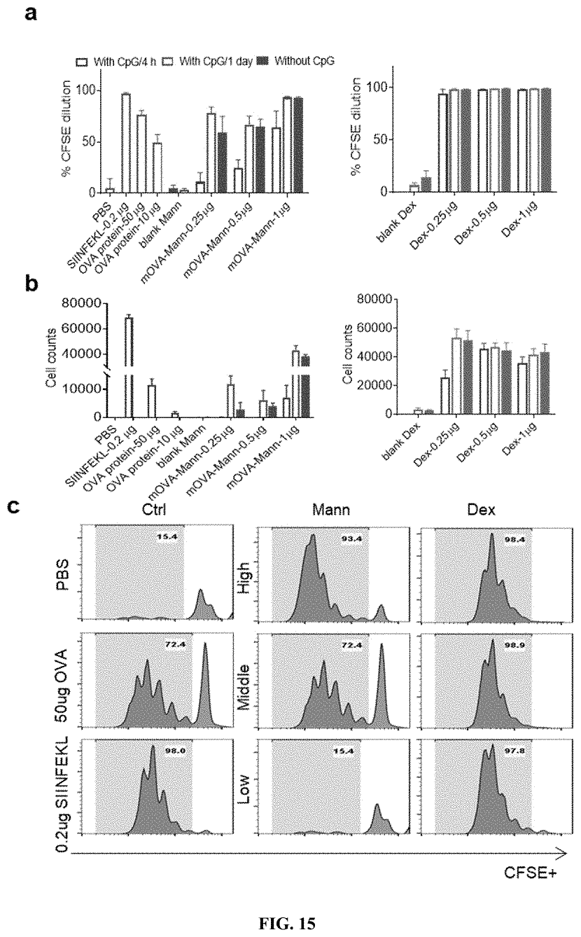

FIG. 16 Proliferation rate of and cytokine release from OT-II T-cells co-cultured with BMDCs with Mann- (a), or Dex-capsules (b) for 24 hr. Representative histograms of CFSE+OT-II T-cells.

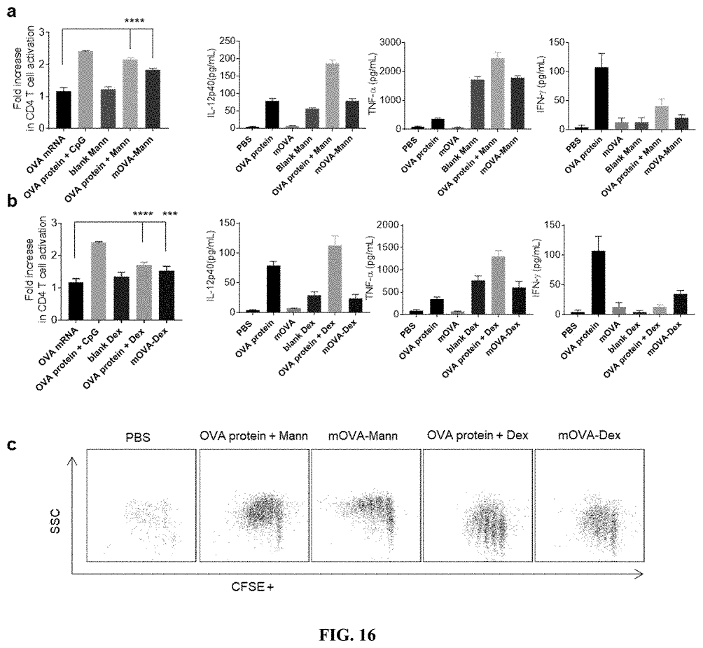

FIG. 17 Optimization of formulation design of nanocapsules. Schematic illustration of nanocapsule formulation process utilizing carboxylated silica nanoparticle as a template (a), and corresponding TEM images of nano-formulations for each formulation step (b). Notably, without chemical crosslinking in the formulation, capsule formation after silica nanoparticle core removal was not complete, resulted in disrupted particle structure as shown in TEM images of (a, 4).

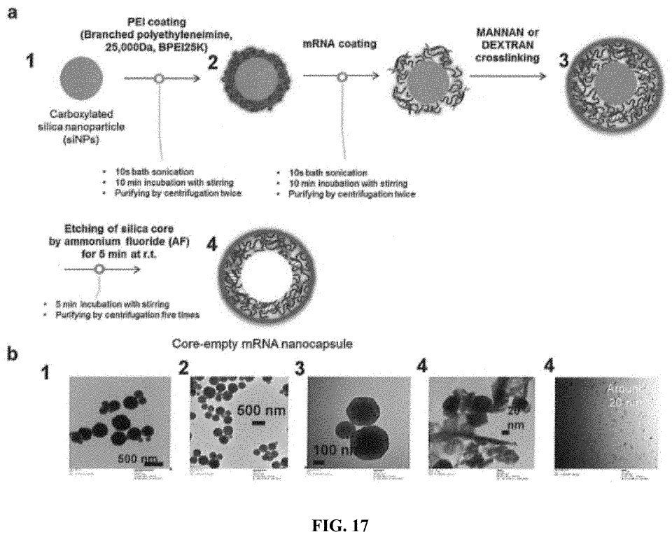

FIG. 18 Formulation and characterizations of nanocapsules made up with various materials using the developed platform technology. TEM images (a, b), AFM images (c,d,e), hydrodynamic sizes and zeta-potentials (f,g) and TEM images of mRNA loaded MANN capsules (h).

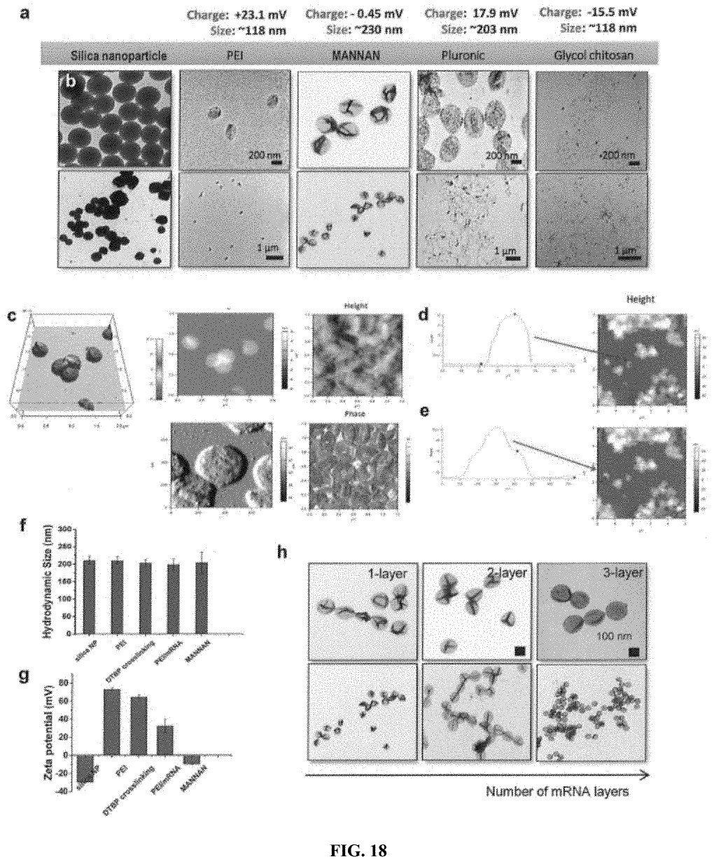

FIG. 19 Adjuvanticity of MANN capsules in vitro BMDC culture. BMDC activation marker staining, cytokine release trend and BMDC maturation image. Multiflex cytokine and chemokine analysis of MANNAN mediated BMDC activation.

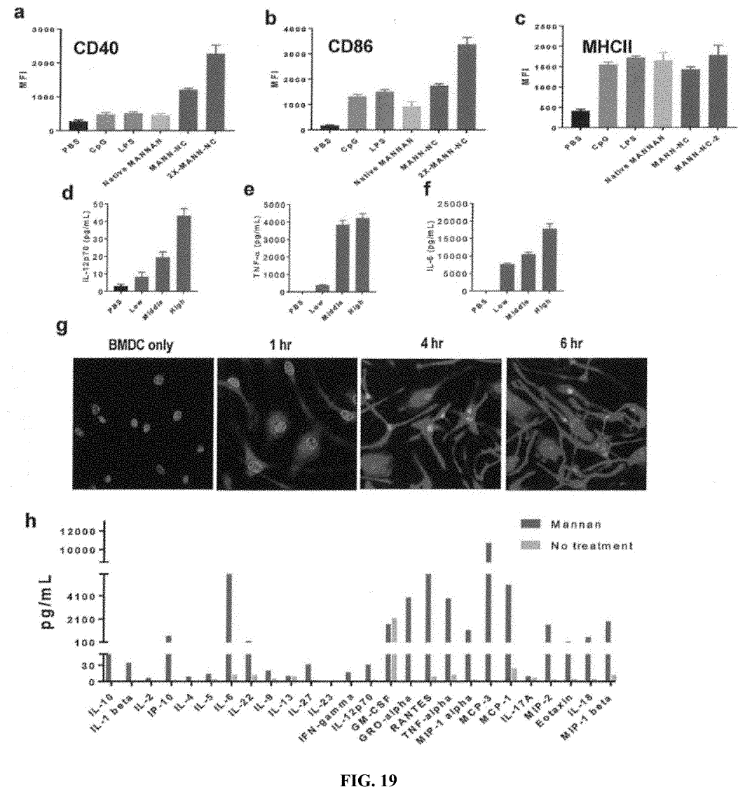

FIG. 20 Mechanistic explanation of how Mannan capsules interact with BMDC compared to Native MANN. Dectin-2 and TLR-4 mediated interaction.

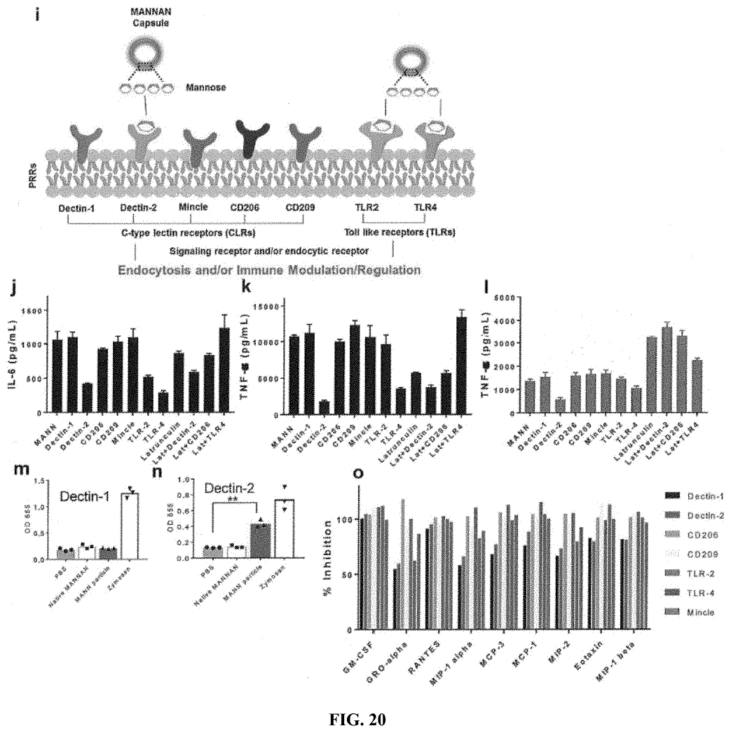

FIG. 21 mRNA uptake in dose and time dependent kinetics of mRNA loaded MANN-NC, cytotoxicity, and transfection in BMDC.

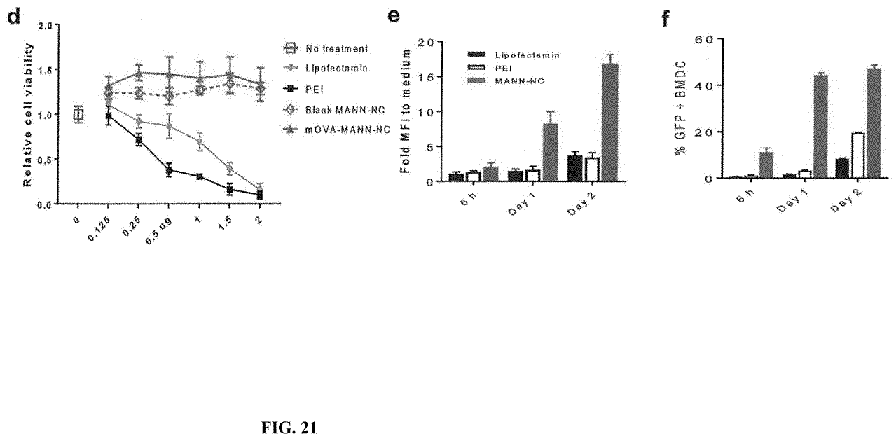

FIG. 22 Antigen presentation ability of MANN-NC by H-2Kd antibody staining in BMDC culture.

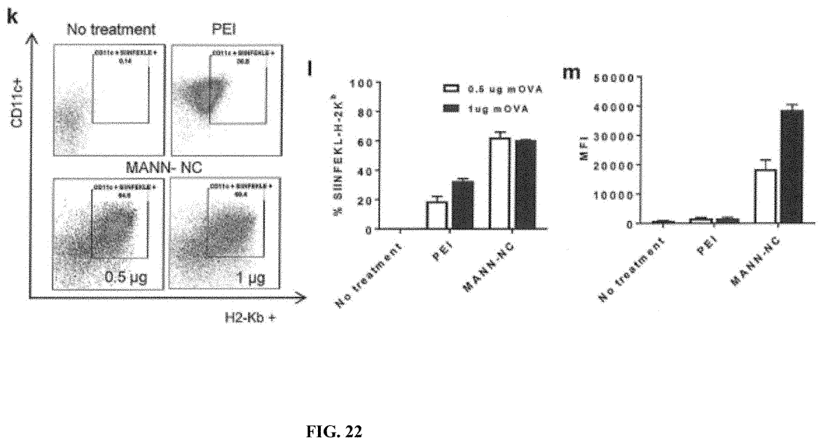

FIG. 23 Cross-priming of CD4 and CD8 T-cells by mRNA encoding OVA antigen loaded into MANN-NC (mOVA-MANN-NC), 2×10⁴ BMDCs were incubated with 1 μg of mOVA loaded MANN-NC formulations including control groups, ovalbumin (OVA) protein (50 μg)/CpG (10 μg) and 1 μg of soluble mOVA for 4 or 24 h. CD4+ and CD8+ T cells from spleen and lymph nodes (LNs) of OT-I and OT-II mice were labelled with 0.1 μM carboxyfluorescein succinimidyl ester (CFSE). The CFSE labelled OVA-specific T cell receptor transgenic CD8+OT-I and CD4+OT-II T cells (1×10⁵) were then cultured with the BMDCs pulsed with various MANN formulations for 3 days in DC media with IL-2 for CFSE dilution. a-c, OT-IT-cells cocultures; Fold increase in CFSE dilution (a), frequency of OT-1 T-cells (b) and representative histograms (c) with BMDC co-cultures with indicated formulations. d-f, OT-II T-cells co-culture; Fold increase in CFSE dilution (d), representative histograms (e) and cytokines levels of IL-12p40 (f), TNF-α (g) and IFN-γ (h) with BMDC co-cultures with indicated formulations.

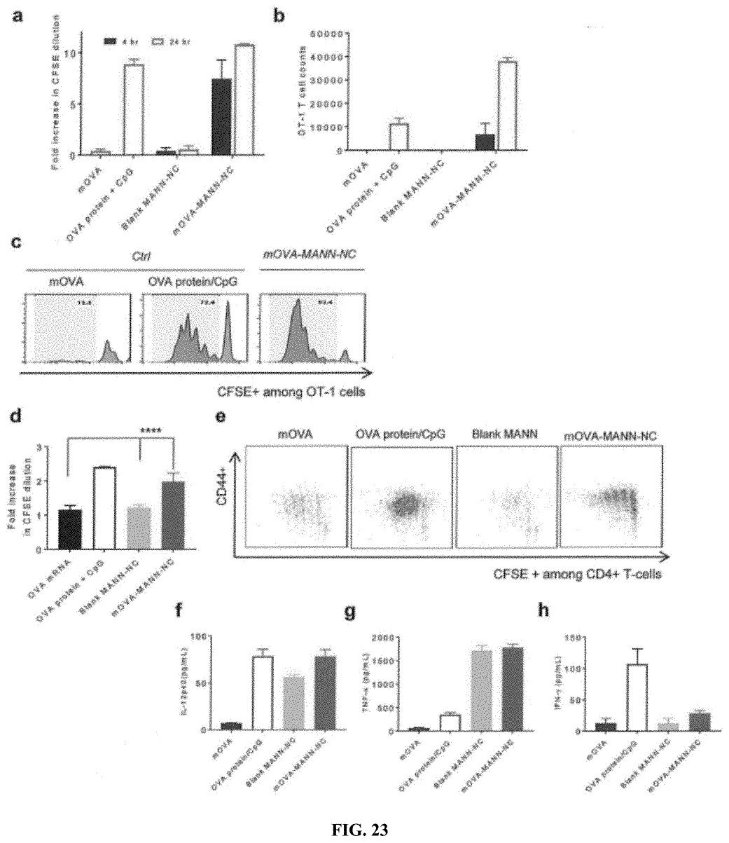

FIG. 24 C57BL/6 mice were immunized with Cy5.5-labelled MANN-NC for 12 hr and LNs were excised for further each immune cell analysis. IVIS images of mice received Cy5.5-labelled MANN-NCs and excised inguinal and auxillary LNs (a) and total radiant efficiency (b). MANN-NC uptake efficiency in each cell subset in LNs (c, d). Cell counts in LNs from mice receiving MANN-NC in comparison to PBS-treated mice (e,f). Activation marker staining of CD86+CD11 c+ DCs, CD80+ Macrophages and CD107+NK cells upon MANN-NC immunization.

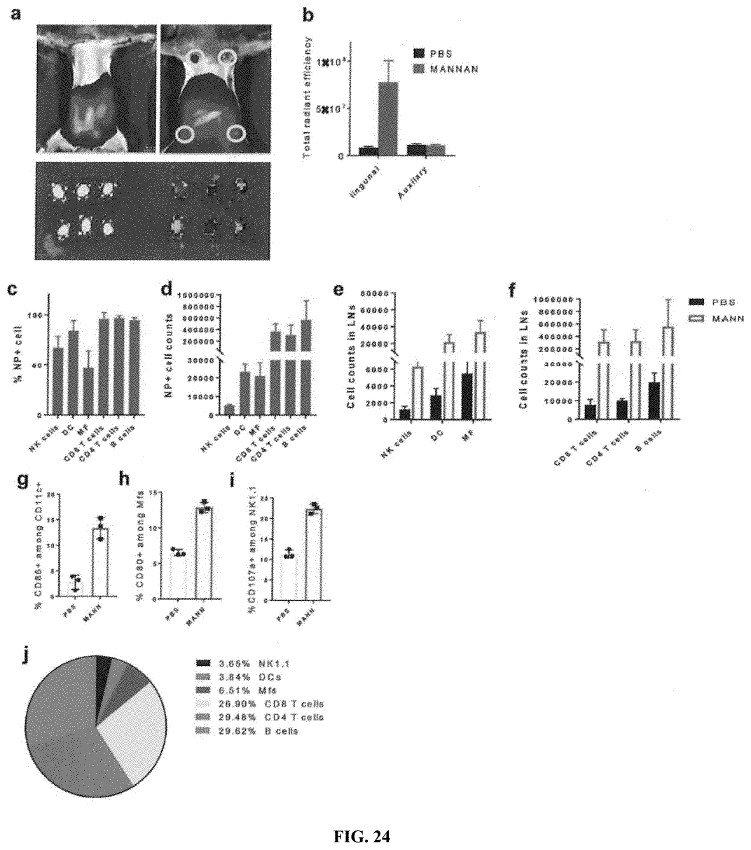

FIG. 25 C57BL/6 mice were inoculated subcutaneously with 2×10⁵B16F100VA cells on day 0. On days 4, 9 and 14, tumor-bearing mice were treated with indicated mRNA-containing formulations at 10 μg mRNA. The average tumor growth (b) and individual tumor growth (c) curves of mice treated with indicated formulations. Data represent mean±SD. n=6. ELISPOT analysis of IFN-γ spot-forming CD8+ and CD4+ T-cells among splenocytes after ex vivo restimulation with SIINFEKL on day 22 (d,e). f-l, Tumor microenvironment analysis on day 26; Tumor infiltrating CD8+ T-cells among CD45+ tumor infiltrating leukocytes (Tl Ls) (f); the frequency of Ag-specific CD8+ T-cell responses (g) and representative scatter plots (h); frequency of CD4+ T cells among CD45+Tl Ls, CD11 c+ DCs of total tumor cells, activated CD86+ DCs and NK1.1+NK cells of total tumor cells. **p<0.01, ***p<0.001, and ****p<0.0001, analyzed by two-way ANOVA with Bonferroni multiple comparisons post-test.

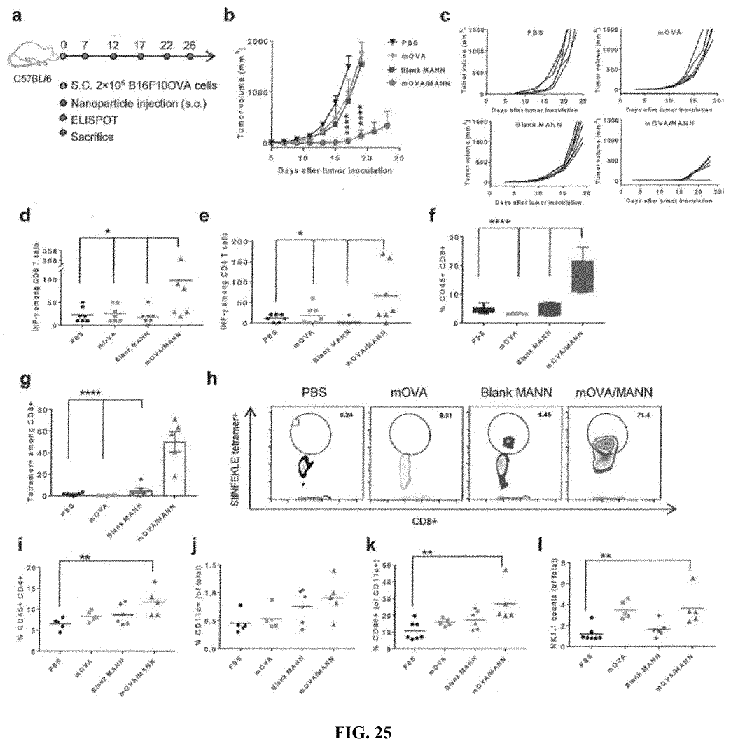

FIG. 26 Adpgk neo-antigen vaccination in MC38 colon carcinoma tumor model. a. C57BL/6 mice were inoculated subcutaneously with 2×105 MC38 cells on day 0. On days 8, 13, 22 and 27, tumor-bearing mice were treated with indicated Adpgk neo-antigen-containing formulations at 10-20 μg Adpgk peptide. The average tumor growth (b) and individual tumor growth (c) curves of mice treated with indicated formulations. Data represent mean±SD. n=6. The frequency of CD8+ T-cells (e) and antigen-specific CD8+ T-cells in peripheral blood on day 19 and 27 (d). *p<0.1, **p<0.01, ***p<0.001, and ****p<0.0001, analyzed by two-way ANOVA with Bonferroni multiple comparisons post-test.

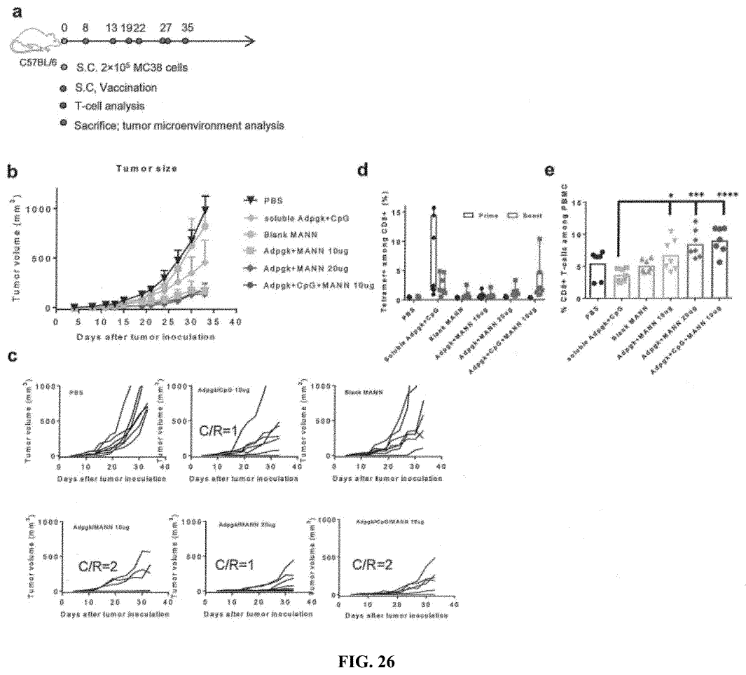

FIG. 27 Tumor microenvironment analysis on day 35; the frequency of NK cells among TILs (f) and representative scatter plots (g); frequency of CD4+ T-cells (h) and Foxp3+ among CD4+ T-cells (i) and representative scatter plots (j).

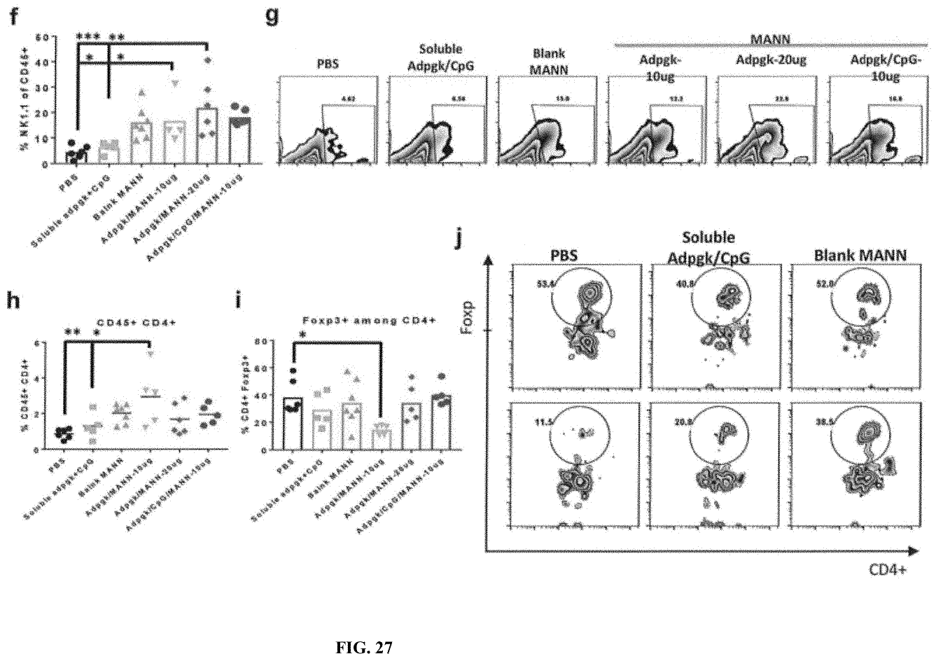

FIG. 28 DEX-NC induced Treg cells.

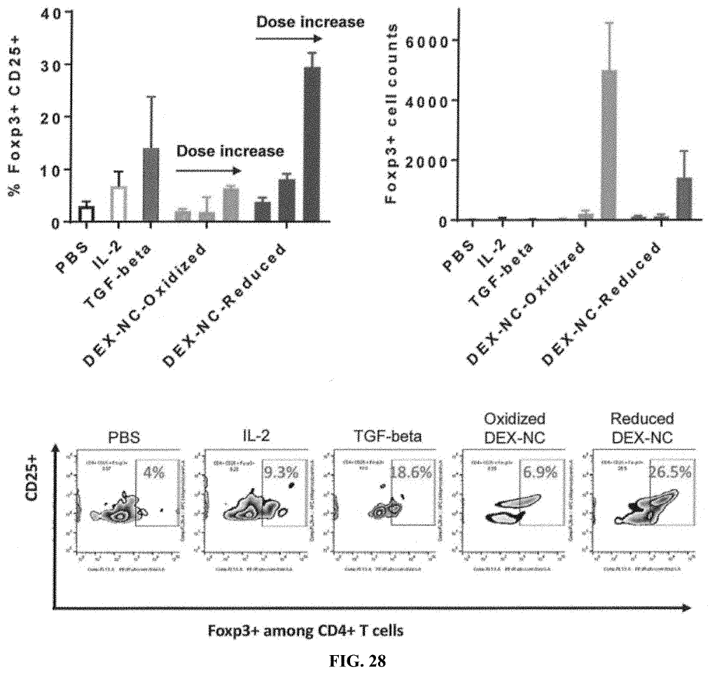

FIG. 29 In vitro mRNA encoding GFP transfection efficiency in BMDC of DEX-NC compared to PEI, which is a conventional transfection agent for gene transfection. Mean florescence intensity of GFP signals (a) % GFP positive BMDCs (b) DEX-NC mediated cross-priming of CD4 and COB T cells by CFSE dilution and cytokine analysis of OT-I (c,d,e) and OT-II (f,g,h,l,j,k) cells.

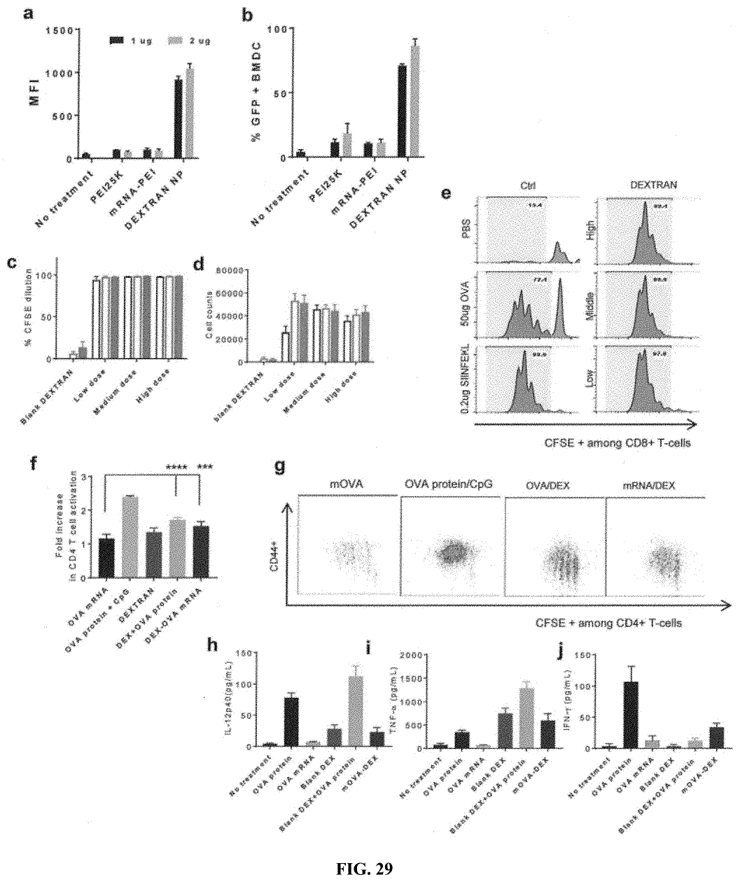

FIG. 30 Lymph nodes draining efficacy and adjuvanticity in immune cell activation in LNs.

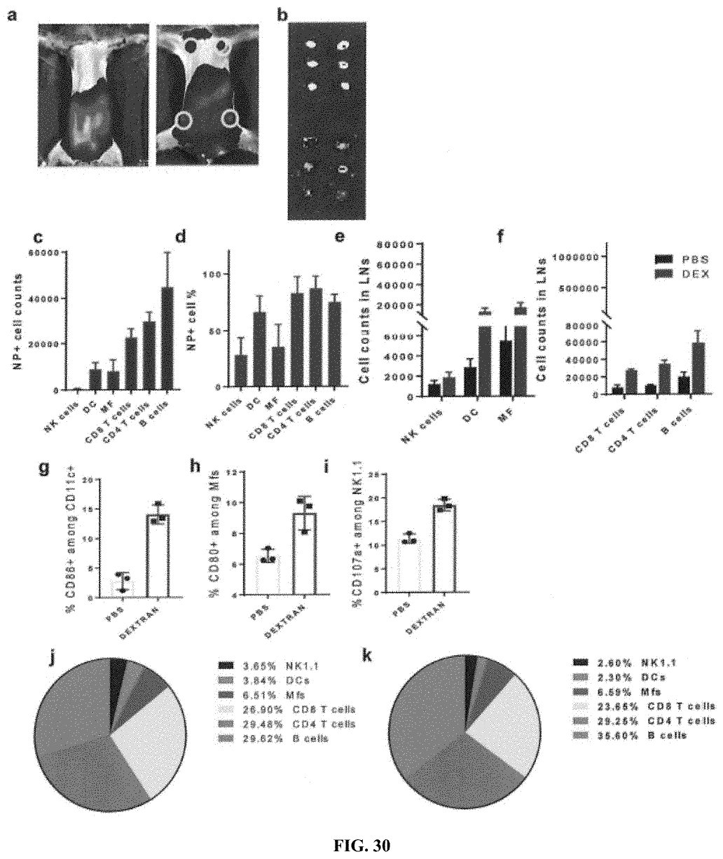

FIG. 31 Anti-tumor therapeutic outcome of mOVA loaded DEX-NC in 816F100VA tumors. Tumor growth rate (a), ELISPOT of CD8 and CD4 T-cells (b,c) Tumor infiltrating CD8-T cells (d) and antigen specific CDBT-cells (e), Tumor infiltrating CD4 T-cells (f).

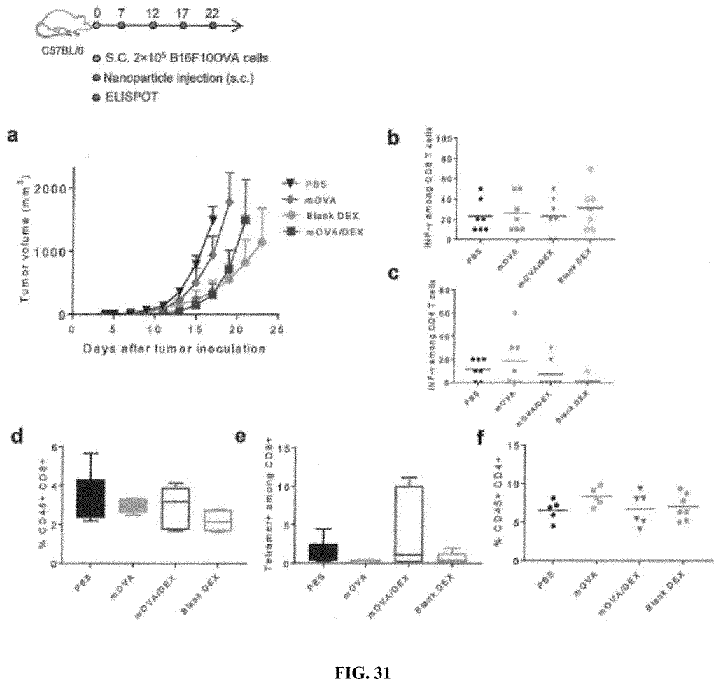

FIG. 32 Antigen-specific CDS T cell responses upon vaccination of DEX/SIINFEKL/CpGNC. Naive C57BL/6 mice were immunized on day 0, 13 and 23, and SIINFEKL tetramer+CD8 T cells were analyzed followed by each immunization.

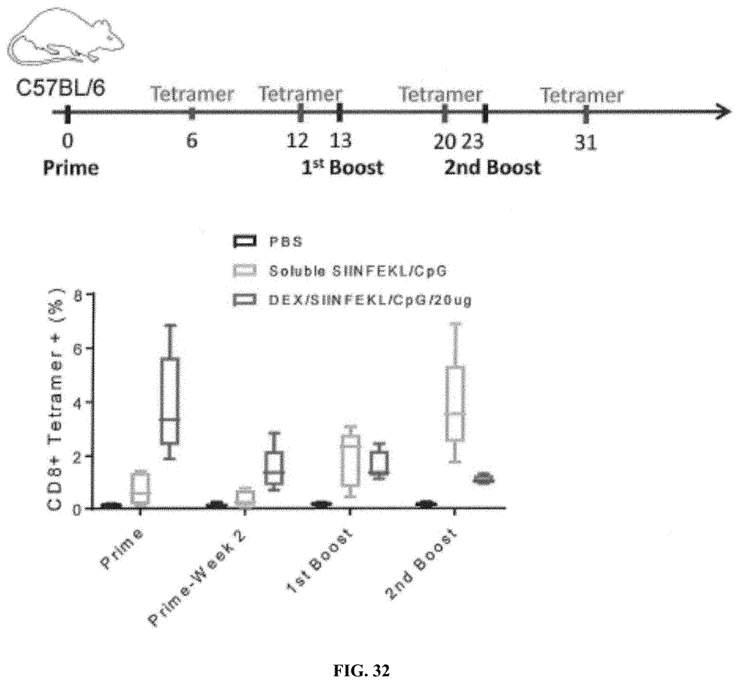

FIG. 33 Antitumor therapeutic efficacy of adpgk/CpG/DEX-NC in MC38 tumors. The mice were immunized on day 7, 14 and 21 after tumor inoculation and antigen-specific CD8+ T cells response were evaluated on day 14, 21 and 28 (a). Tumor growth rate (b) survival rate (c) and tetramer staining against adpgk (c).

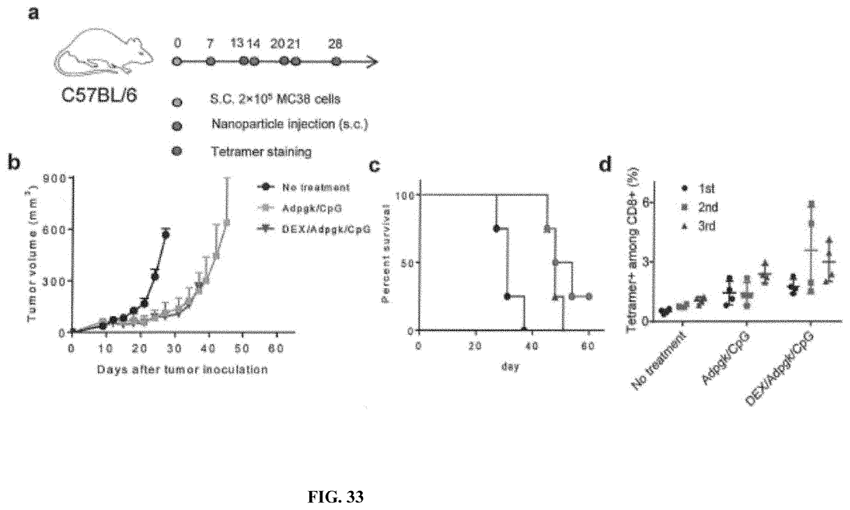

FIG. 34 contains a scheme illustrating mechanism of activation of innate immunity of a tumor by Manna-based nanocapsules.

FIG. 35 Mann-capsule mediated antitumor effect in CT-26 colon carcinoma tumor model. (a) CT-26 tumor was subcutaneously (s.c.) inoculated in BALB/c mice on day 0 and Mann-capsule was intratumorally (i.t.) given on day 9, 12 and 15 for average tumor growth rate (b), individual tumor growth rate (d) and survival rate (c). Cytokine and chemokine levels in blood (f) and tumors (g) by i.t. Mann-capsule injection were measured as indicated time point in scheme (e).

FIG. 36 Antibody depletion study. Respective immune cell subset in mice received Mann-capsule intratumorally was depleted by depletion antibody by four times intraperitoneal injections in CT26 tumor bearing mice. Average tumor growth curve (a) and survival rate (b). CD8 T-cells and NK cells are mainly responsible for Mann-capsule mediated tumor growth control.

FIG. 37 Tumor microenvironment analysis of CT-26 tumor received Mann-capsule. Frequencies of CD8+ T-cells and tetramer+CD8 T-cells (c) and their representative histograms (d). Frequency of CD4+ T-cells among whole tumor cells and representative histograms (e); frequency of Foxp3+CD4 T-cells and representative histogram (f) CD206 expression level (mean fluorescence intensity and frequency) among F480+macrophages among whole tumor cells (g); NKG2A expression level on CD8 T-cells and NK cells. Taking all into account, Mann-capsule render tumor microenvironment significantly less immunosuppressive.

FIG. 38 Combination study in CT26 tumor-bearing BALB/c mice with OX40 antibody. Mann-capsule in combination with anti-OX40 regressed established CT26 tumor mass and extended survival rate (a) significantly with three intratumoral injections as indicated above. Systemic immune activation by local treatment of Mann-capsule were evaluated by intracellular cytokine staining of CD8 T-cells (b) and NK cells (c) and ELISPOT analysis (d) among splenocytes after restimulation with AH1 antigen peptide.

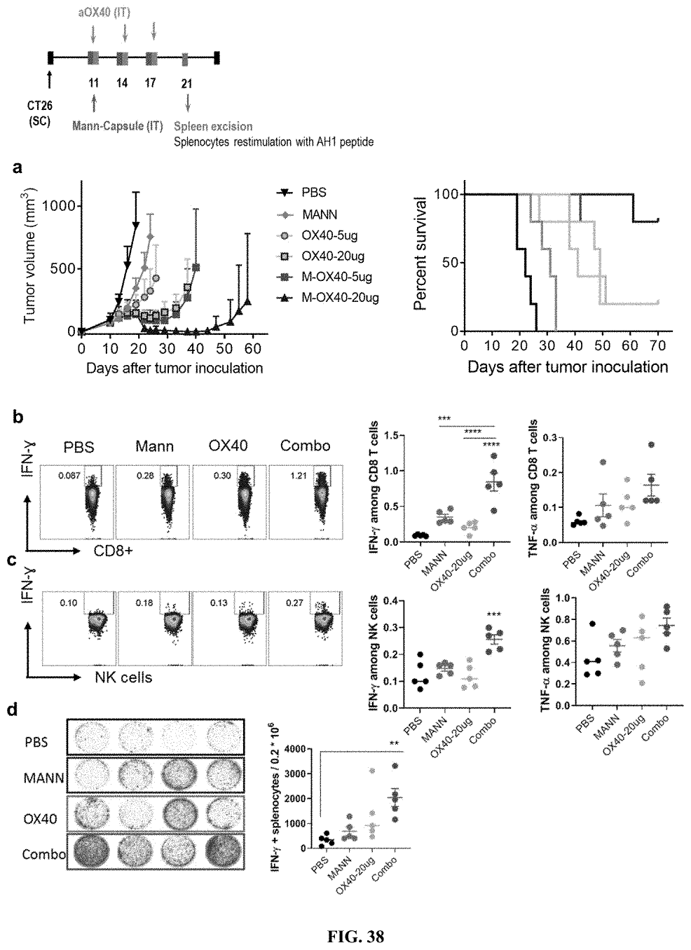

### DETAILED DESCRIPTION

Antigen presenting cells (APCs), such as dendritic cells (DCs), recognize the distinct molecular patterns in polysaccharides present on the surface of various pathogens. In particular, polysaccharides on bacterial and fungal cell walls are known to trigger strong DC activation. The present disclosure is related to polysaccharide-coated nanocapsules that can target and activate DCs for intracellular delivery of various therapeutic biomolecules (payload), such as tumor antigen-encoding messenger RNA (mRNA). The results described herein demonstrate that polysaccharide-coated capsule not only targets the cell, but also protects the payload (e.g., mRNA) from enzymatic degradation, mediates its efficient uptake by the cell, and improves translation of mRNA and activation of the DC. Notably, the hollow core of the nanocapsule facilitates efficient drainage of the lymph nodes, significantly improving delivery of the payload (e.g., mRNA) to DCs in lymph nodes. In one non-limiting example, the nanocapsule, composed of mannan, carrying ovalbumin-encoding mRNA as the payload, elicits robust antigen-specific CD8+ T-cell response, leading to significant reduction in tumor burden in tumor bearing-mice. Various embodiments of the nanocapsules, the methods of making the nanocapsules, and the methods of using the nanocapsules as adjuvants, vaccines, and delivery vehicles for the therapeutic biomolecules, are described herein.

Particles

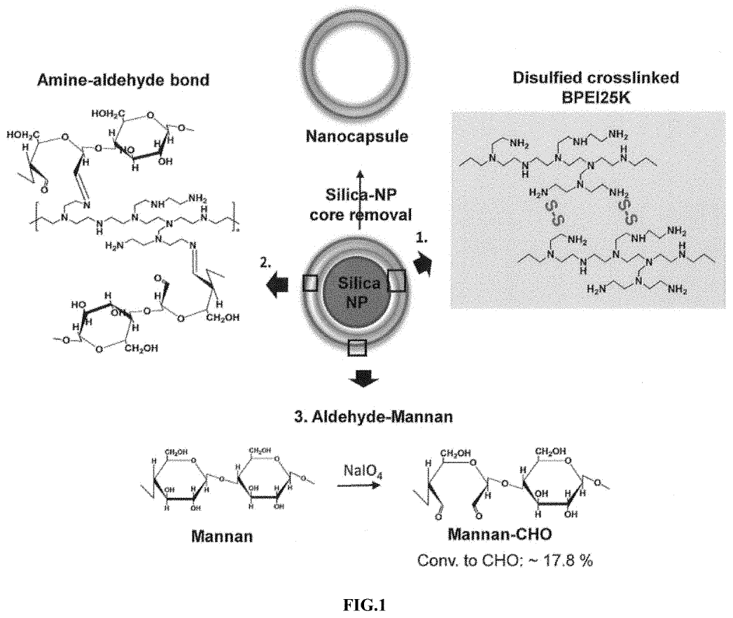

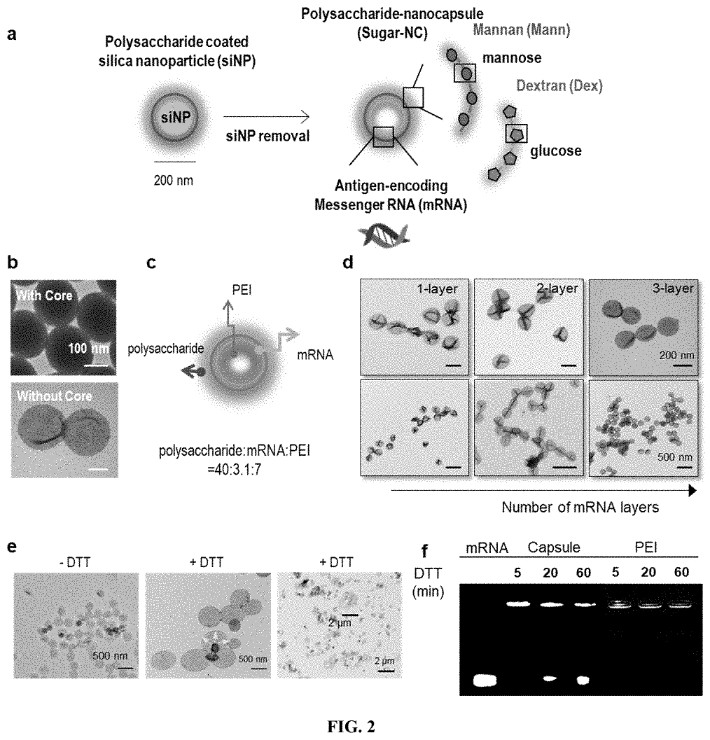

Referring to FIGS. 1 and 2 herein, the particles disclosed in this application comprise a hollow core and a shell surrounding the hollow core. The shell comprises a polyamine polymer layer, for example, a crosslinked polyamine polymer layer, which layer is also coated with a polysaccharide coating. In some embodiments, the particle also comprises a biomolecule between the polyamine layer and the polysaccharide coating.

The core of the particle is hollow, that is, in some embodiments, there is no matter at all in the core of the particle other than air or a solvent (e.g., water, saline, dextrose solution, or a buffer). In some embodiments, the core of the particle comprises trace amounts of silica, trace amounts of fluoride source, or an amount of a solvent, such as water or a buffer. In some embodiments, the core of the particle comprises trace amounts of a biomolecule, or trace amounts of a crosslinker. In certain embodiments, the core of the particle comprises water and no other matter. Because the core is hollow, the particle of the present disclosure is also referred to as a “capsule.”

In some embodiments, the size of the particle is from about 200 nm to about 400 nm (i.e., the particle is a nanoparticle), or from about 200 nm to about 300 nm. Such nanoparticle is referred to herein as a “nanocapsule.” In some embodiments, the thickness of the shell of the nanocapsule is from about 1 nm to about 50 nm, or from about 5 nm to about 15 nm. In some embodiments, the zeta-potential of the particle is from about −50 mV to about 5 mV, of from about −40 mV to about 1 mV In some embodiments, the particle is negatively charged and the zeta-potential is from about −35 mV to about −50 mV, or from about −40 mV to about −45 mV. In some embodiments, the zeta potential is from about −10 mV to about −100 mV, from about −20 mV to about −80 mV, from about −30 mV to about −70 mV, or from about −40 mV to about −60 mV In some embodiments, the zeta potential is about −10 mV, about −20 mV, about −30 mV, about −40 mV, about −45 mV, or about −50 mV.

In some embodiments, the particles present within a population, e.g., in a composition, can have substantially the same shape and/or size (i.e., they are “monodisperse”). For example, the particles can have a distribution such that no more than about 5% or about 10% of the particles have a diameter greater than about 10% greater than the average diameter of the particles, and in some cases, such that no more than about 8%, about 5%, about 3%, about 1%, about 0.3%, about 0.1%, about 0.03%, or about 0.01% have a diameter greater than about 10% greater than the average diameter of the particles. In some embodiments, the diameter of no more than 25% of the particles varies from the mean particle diameter by more than 150%, 100%, 75%, 50%, 25%, 20%, 10%, or 5% of the mean particle diameter. It is often desirable to produce a population of particles that is relatively uniform in terms of size, shape, and/or composition so that most of the particles have similar properties. For example, at least 80%, at least 90%, or at least 95% of the particles produced using the methods described herein can have a diameter or greatest dimension that falls within 5%, 10%, or 20% of the average diameter or greatest dimension. In some embodiments, a population of particles can be heterogeneous with respect to size, shape, and/or composition. In some embodiments, the polydispersity index is from about 0.01 to about 0.5, from about 0.05 to about 0.5, from about 0.1 to about 0.4, or from about 0.1 to about 0.3. In some embodiments, the polydispersity index is about 0.05, about 0.1, about 0.15, about 0.2, about 0.25 or about 0.3.

Polyamine

In some embodiments, polyamine of the particle of the present disclosure is a polymer comprising multiple amine units. In one example, the polyamine is a polyester polyamine copolymer. In some embodiments, the polyester component of such polymer is selected from the group consisting of poly(glycolic acid), poly(lactic acid), poly(caprolactone), and poly(lactide-co-glycolide). In some embodiments, the polyamine component of the copolymer may include C2-6 alkylene diamine, such as ethylene diamine, propylene diamine, butylene diamine, or a polyamine compound having more than two amino groups, such as spermidine, spermine, tris(2-aminoethyl)amine, thermospermine, diethylenetriamine (DETA), triethylenetetramine (TETA), tetraethylenepentamine (TEPA), pentaethylenehexamine (PEHA). In some embodiments, the polyamine polymer contains poly(ethylene glycol) as one of the components.

In some embodiments, the amine compound is a polyethyleneimine, or a salt thereof. In some embodiments, the polyethyleneimine is linear. In other embodiments, the polyethyleneimine is branched (e.g., CAS Reg. Nos. 9002-98-6, 25987-06-8). For example, a polyethyleneimine can have from 2 to 100 termini (e.g., 2 to 80, 2 to 75, 2 to 60, 2 to 50, 2 to 40, 2 to 35, 2 to 25, 2 to 10, 2 to 5, 4 to 20, 5 to 25, 10 to 50, 25 to 75, 3 to 6, 5 to 15 termini). In some embodiments, a polyethyleneimine can have from 2 to 5, 4 to 6, 5 to 6, or 3 to 6 termini. In some embodiments, branched polyethyleneimine is V-shaped or T-shaped, depending on the method by which polyethyleneimine has been synthesized. In some embodiments, the polyethyleneimine has both linear and branched fragments. In some embodiments, the polyethyleneimine is alkylated (e.g., methylated or ethylated). In some embodiments, the polyethyleneimine is PEGylated (e.g. reacted with ethylene oxide to form PEG chains having molecular weight between about 1,000 Da and about 100,000 Da).

In some embodiments, polyethyleneimine has average molecular weight between about 0.1 kDa to about 500 kDa. For example, molecular weight of polyethyleneimine may be between about 500 Da and about 100,000 Da. Polyethyleneimine described herein can have a molecular weight of about 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, or 500 Da. In some embodiments, the molecular weight of polyethyleneimine is between about 500 Da and about 50,000 Da. In some embodiments, molecular weight of polyethyleneimine is between about 500 Da and about 40,000 Da. In some embodiments, molecular weight of polyethyleneimine is between about 1,000 Da and about 40,000 Da. In some embodiments, molecular weight of the polyethyleneimine is between about 5,000 Da and about 40,000 Da. In some embodiments, molecular weight of polyethyleneimine is between about 10,000 Da and about 40,000 Da. An exemplary fragment of a polyethyleneimine polymer is shown in FIG. 1.

Without being bound by any particular theory, it is believed that the polyamine covalently reacts with the silica (e.g., carboxylated silica) of the particle template (as discussed in the methods of making section herein), and also with the aldehyde groups of the polysaccharide in the polysaccharide coating (discussed more fully under the “polysaccharide” subsection herein). The polyamine layer provides structural backbone and structural support to the particle. The crosslinking of the polyamine further reinforces the structural stability and rigidity of the polyamine layer, providing better support for the particle. In one embodiment, when polyamine is not crosslinked, the particle breaks down and “collapses” during the final steps of the preparation of the particle.

Crosslinker

Any suitable crosslinker can be used to crosslink the polyamine layer of the particle during its preparation. In one example, a crosslinker contains a —S—S— bridge. Suitable examples of such crosslinkers include:

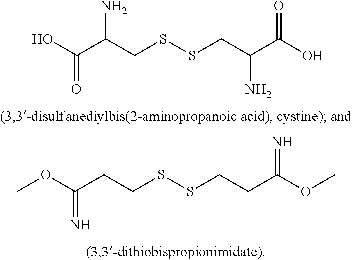

Other suitable examples of crosslinkers include bis-functional compounds capable of chemically reacting with the amine groups of polyamine. Suitable examples of bis-functional compounds include dialdehides and diacids, among other classes of compounds. In some embodiments, the crosslinker is selected from adipic, sebacic, malonic, succinic, glutaric, pimelic, suberic, and azelaic acid. In some embodiments, the crosslinker is succinaldehyde or glutaric dialdehyde. Without being bound by any theory, it is believed that the —S—S— bridge containing crosslinker is reduced by intracellular reductases when the particle is phagocyted by the antigen-presenting cell, thereby contributing to the collapse and hydrolysis of the particle. This way, the biomolecule is released from the particle and is further processed by the APC. In a similar manner, diacid and dialdehyde crosslinker can be hydrolyzed by intracellular hydrolases to collapse the particle and release the biomolecule. An example of crosslinking of polyethyleneimine with a —S—S— containing crosslinker is shown in FIG. 1.

Polysaccharide

In some embodiments, the polysaccharide is positively charged. In other embodiments, the polysaccharide is negatively charged. The polysaccharide may be a natural or synthetic polysaccharide comprising any number of monosaccharide residues (including any acyclic and/or cyclic forms and any possible stereoisomers) selected from allose, altrose, arabinose, erythrose, erythrulose, fructose, fucosamine, fucose, galactosamine, galactose, glucosamine, glucosaminitol, glucose, glyceraldehyde, glycerol, glycerone, gulose, idose, lyxose, mannosamine, mannose, psicose, quinovose, quinovosamine, rhamnitol, rhamnosamine, rhamnose, ribose, ribulose, sorbose, tagatose, talose, threose, xylose, xylulose, glucuronic acid and derivatives thereof. In some embodiments, monosaccharide (sugar) residues in the polysaccharide are selected from the group consisting of glucose, fructose, galactose, mannose, ribose, arabinose, xylose, N-acetylglucosamine, glucuronic acid, glucosamine, sialic acid, iduronic acid, galactosamine, and derivatives thereof. In some embodiments, the polysaccharide is glycosaminoglycan, such as heparin, heparan sulfate, chondroitin sulfate, dermatan sulfate, keratan sulfate, or hyaluronic acid. In some embodiments, the polysaccharide is polyglucosamine such as chitosan. In some embodiments, the polysaccharide is mannan (linear polymer of the sugar mannose). In some embodiments, the polysaccharide is dextran (polysaccharide derived from the condensation of glucose).

In some embodiments, the polysaccharide comprises a pathogen-associated molecular pattern (PAMP), which is a molecular fragment of a polysaccharide that corresponds to a molecule or a part of a molecule obtained from a pathogen (e.g., bacteria, virus, fungus, or a cancer cell). In some embodiments, the PAMP is a fragment of a bacterial or fungal cell wall. Suitable examples of PAMP include bacterial lipopolysaccharides (LPSs), endotoxins (e.g., endotoxins found on the cell membranes of gram-negative bacteria), bacterial flagellin, lipoteichoic acid (e.g., from gram-positive bacteria), peptidoglycans, and nucleic acid variants associated with viruses, such as double-stranded RNA (dsRNA) and unmethylated CpG (cytosine triphosphate deoxynucleotide-phosphodiester-guanine triphosphate deoxynucleotide) oligodeoxynucleotide motifs.

In some embodiments, the polysaccharide is oxidized. In some examples the oxidized polysaccharide contains at least one aldehyde group (C═O)H. Such polysaccharides can be obtained, for example, when some of the monosaccharide units within the polysaccharide containing a vicinal diol system (C(OH)—C(OH)) are oxidized to produce a dialdehyde, for example, as shown in FIG. 1. Suitable examples of oxidizing agents include NaIO₄ and KMnO₄. In some embodiments, the oxidized aldehyde contains from about 1 wt. % to about 25 wt. % of aldehyde groups, for example, about 1 wt. %, about 2 wt. %, about 2.5 wt. %, about 5 wt. %, or about 10 wt. %. In some embodiments, the polysaccharide is oxidized mannan or an oxidized dextran (CHO-MANN or CHO-DEX). Without being bound by a theory, it is believed that the CHO groups covalently react with the amino groups of the polyamine and therefore facilitate bonding of the polysaccharide coating to the polyamine layer of the particle.

In some embodiments, the polysaccharide comprises a tumor-targeting moiety, such as an RGD peptide or a CD27 antibody or CD20 antibody.

Therapeutic Biomolecules

In some embodiments, biomolecules are organic molecules having a molecular weight of 200 daltons or more produced by living organisms or cells, including large polymeric molecules such as polypeptides, proteins, glycoproteins, polysaccharides, polynucleotides and nucleic acids, or analogs or derivatives of such molecules.

Therapeutic Proteins and Peptides

In some embodiments, the biomolecule is a therapeutic protein or peptide, such as an antibody, a hormone, a transmembrane protein, a growth factor, an enzyme, or a structural protein.

In some embodiments, therapeutic protein is a therapeutic peptide (e.g., containing 50 or fewer amino acids, 40 or fewer amino acids, 30 or fewer amino acids, 20 or fewer amino acids, or any number of amino acids that does not exceed 50). In some embodiments, therapeutic peptide is Cpd86, ZPGG-72, ZP3022, MOD-6030, ZP2929, HM12525A, VSR859, NN9926, TTP273/TTP054, ZYOG1, MAR709, TT401, HM11260C, PB1023, ZP1848, ZP4207, ZP2929, Dulaglutide, Semaglutide, or ITCA. In some embodiments, therapeutic peptide in any one of peptides described in Fosgerau et al. Drug Discovery Today, Volume 20, Issue 1, January 2015, Pages 122-128, Kaspar et al., Drug Discovery Today, Volume 18, Issues 17-18, September 2013, Pages 807-817, the disclosures of which are incorporated herein by reference in their entirety.

In some embodiments, the therapeutic peptide is negatively charged.

In some embodiments, the therapeutic protein is negatively charged, e.g., the therapeutic protein comprises a functional group that is negatively charged as physiological pH, e.g., acids, including carboxylic acids (carboxylates), sulfonic acids (sulfonates), sulfates, and phosphonates.

In some embodiments, the protein therapeutic is any one of protein therapeutics described in, e.g., Leader et al., Nature Reviews 2008, 7, 21-39, the disclosure of which is incorporated herein by reference in its entirety.

In some embodiments, the therapeutic protein is a cytokine, such as transforming growth factor-beta (TGF-beta), interferons (e.g., interferon-alpha, interferon-beta, interferon-gamma), colony stimulating factors (e.g., granulocyte colony stimulating factor (GM-CSF)), and thymic stromal lymphopoietin (TSLP).

In some embodiments, the interferon is interferon-αcon1, interferon-alpha2a, interferon-α2b, interferon-αn3, interferon-β1a, or interferon-γ1b.

In some embodiments, the cytokine is an interleukin, such as interleukin-1, interleukin-2, interleukin-3, interleukin-4, interleukin-5, interleukin-6, interleukin-7, interleukin-8, interleukin-10, interleukin-12, interleukin-13, interleukin-15, interleukin-17, interleukin-18, interleukin-22, interleukin-23, and interleukin-35.

In some embodiments, the therapeutic protein is a polypeptide hormone, such as amylin, anti-Müllerian hormone, calcitonin, cholecystokinin, corticotropin, endothelin, enkephalin, erythropoietin (EPO), darbepoetin, follicle-stimulating hormone, gallanin, gastrin, ghrelin, glucagon, gonadotropin-releasing hormone, growth hormone-releasing hormone, hepcidin, human chorionic gonadotropin, growth hormone (GH), human growth hormone (hGH), inhibin, insulin, isophane insulin, insulin detemir, insulin glargine, pramlintide, pramlintide acetate, insulin-like growth factor, leptin, luteinizing hormone, luteinizing hormone releasing hormone, melanocyte stimulating hormone, motilin, orexin, oxytocin, pancreatic polypeptide, parathyroid hormone, prolactin, secretin, somatostatin, thrombopoietin, thyroid-stimulating hormone, vasoactive intestinal peptide, somatotropin, mecasermin, mecasermin rinfabate, human follicle-stimulating hormone, lutropin, teriparatide, exenatide, octreotide, dibotermin-α, bone morphogenetic protein 7, keratinocyte growth factor, platelet-derived growth factor, trypsin, nesiritide and vasopressin.

In some embodiments, the therapeutic protein is factor VIIa, factor VIII, factor IX, antithrombin III, protein C, drotrecogin-α, filgrastim, pegfilgrastim, sargramostim, Lepirudin, Bivalirudin, or oprelvekin.

In some embodiments, the therapeutic protein is botulinium toxin type A, botulinium toxin type B.

In some embodiments, the polypeptide hormone is useful in treating endocrine disorders (hormone deficiencies). In some embodiments, the polypeptide hormone is useful in treating haemostasis and thrombosis.

In some embodiments, the therapeutic protein is an enzyme. In some embodiments, the enzyme is agalsidase beta, imiglucerase, velaglucerase alfa, taliglucerase, alglucosidase alfa, laronidase, idursulfase, β-gluco-cerebrosidase, alglucosidase-α, laronidase, α-L-iduronidase, idursulphase, iduronate-2-sulphatase, galsulphase, agalsidase-β, human α-galactosidase A, α-1-proteinase, α-1-proteinase inhibitor, pancreatic enzyme, lactase, lipase, amylase, protease, adenosine deaminase, alteplase, reteplase, tenecteplase, urokinase, collagenase, human deoxyribonuclease I, dornase-α, hyaluronidase, papain, asparaginase (e.g. L-Asparaginase), rasburicase, streptokinase, anistreplase, or galsulfase.

In some embodiments, the enzyme is useful in treating metabolic enzyme deficiencies. In some embodiments, the enzyme is useful in treating pulmonary and gastrointestinal-tract disorders. In some embodiments, the enzyme is useful in treating immunodeficiencies.

In some embodiments, the therapeutic protein is albumin, human albumin, or immunoglobulin.

In some embodiments, the therapeutic protein is an antibody (e.g., monoclonal antibodies, e.g., bispecific monoclonal antibodies), including therapeutic antibodies.

In some embodiments, the antibody is useful in treating cancer. In some embodiments, the antibody useful in treating cancer is abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, or zalutumumab.

In some embodiments, the antibody is useful in treating an inflammatory disease or condition. In some embodiments, the antibody useful in treating an inflammatory disease or condition is adalimumab, alemtuzumab, atlizumab, basiliximab, canakinumab, certolizumab, certolizumab pegol, daclizumab, muromonab, efalizumab, fontolizumab, golimumab, infliximab, mepolizumab, natalizumab, omalizumab, ruplizumab, ustekinumab, visilizumab, zanolimumab, vedolizumab, belimumab, otelixizumab, teplizumab, rituximab, ofatumumab, ocrelizumab, epratuzumab, eculizumab, or briakinumab.

In some embodiments, the therapeutic protein in useful in treating infectious disease. In some embodiments, the therapeutic protein useful in treating infectious disease is enfuvirtide.

In some embodiments, the therapeutic protein is abciximab, pegvisomant, crotalidae polyvalent immune Fab, digoxin immune serum Fab, ranibizumab, or ordenileukin diftitox.

In some embodiments, the therapeutic protein is useful in treating endocrine disorders (hormone deficiencies). In some aspects of these embodiments, the therapeutic protein is useful in treating diabetes, diabetes mellitus, diabetic ketoacidosis, hyperkalaemia, hyperglycemia, growth failure due to GH deficiency or chronic renal insufficiency, Prader-Willi syndrome, Turner syndrome, AIDS wasting or cachexia with antiviral therapy, growth failure in children with GH gene deletion or severe primary IGF1 deficiency, postmenopausal osteoporosis, severe osteoporosis, type 2 diabetes resistant to treatment with metformin and a sulphonylurea, or acromegaly.

In some embodiments, the therapeutic protein is useful in treating haemostasis and thrombosis. In some aspects of these embodiments, the therapeutic protein is useful in treating haemophilia A, haemophilia B, hereditary AT-III deficiency in connection with surgical or obstetrical procedures or for thromboembolism, venous thrombosis and purpura fulminans in patients with severe hereditary protein C deficiency, pulmonary embolism, myocardial infarction, acute ischaemic stroke, occlusion of central venous access devices, acute myocardial infarction, haemorrhage in patients with haemophilia A or B and inhibitors to factor VIII or factor IX, severe sepsis with a high risk of death, heparin-induced thrombocytopaenia, blood-clotting risk in coronary angioplasty, acute evolving transmural myocardial infarction, deep vein thrombosis, arterial thrombosis, occlusion of arteriovenous cannula, and thrombolysis in patients with unstable angina.

In some embodiments, the therapeutic protein is useful in treating metabolic enzyme deficiencies. In some aspects of these embodiments, the therapeutic protein is useful in treating Gaucher's disease, Pompe disease, glycogen storage disease type II, Hurler and Hurler-Scheie forms of mucopolysaccharidosis I, mucopolysaccharidosis II, Hunter syndrome, mucopolysaccharidosis VI, or Fabry disease.

In some embodiments, the therapeutic protein is useful in treating pulmonary and gastrointestinal-tract disorders. In some aspects of these embodiments, the therapeutic protein is useful in treating congenital α-1-antitrypsin deficiency, gas, bloating, cramps and diarrhea due to inability to digest lactose, cystic fibrosis, chronic pancreatitis, pancreatic insufficiency, post-Billroth II gastric bypass surgery, pancreatic duct obstruction, steatorrhoea, poor digestion, gas, or bloating.

In some embodiments, the therapeutic protein is useful in treating immunodeficiencies. In some aspects of these embodiments, the therapeutic protein is useful in treating severe combined immunodeficiency disease due to adenosine deaminase deficiency or primary immunodeficiencies.

In some embodiments, the therapeutic protein is useful in treating haematopoiesis. In some aspects of these embodiments, the therapeutic protein is useful in treating anaemia, myleodysplasia, anaemia due to renal failure or chemotherapy, preoperative preparation, anaemia in patients with chronic renal insufficiency and chronic renal failure (+/− dialysis), neutropaenia, neutropaenia in AIDS or post-chemotherapy or bone marrow transplantation, severe chronic neutropaenia, leukopaenia, myeloid reconstitution post-bone-marrow transplantation, HIV/AIDS, thrombocytopaenia (especially after myelosuppressive chemotherapy).

In some embodiments, the therapeutic protein is useful in treating infertility. In some aspects of these embodiments, the therapeutic protein is useful in assisted reproduction and treating infertility with luteinizing hormone deficiency.

In some embodiments, the therapeutic protein is useful in immunoregulation. In some aspects of these embodiments, the therapeutic protein is useful in treating chronic hepatitis C infection, hairy cell leukemia, chronic myelogenous, leukemia, Kaposi's sarcoma, hepatitis B, melanoma, Kaposi's sarcoma, follicular lymphoma, hairy-cell leukemia, condylomata acuminata, hepatitis C, condylomata acuminata (genital warts, caused by human papillomavirus), multiple sclerosis, chronic granulomatous disease, severe osteopetrosis, metastatic renal cell cancer, or melanoma.

In some embodiments, the therapeutic protein is useful in treating diseases or conditions associated with growth regulation. In some aspects of these embodiments, the therapeutic protein is useful in treating acromegaly, symptomatic relief of VIP-secreting adenoma and metastatic carcinoid tumours, spinal fusion surgery, bone injury repair, tibial fracture nonunion, lumbar, spinal fusion, precocious puberty, severe oral mucositis in patients undergoing chemotherapy or debridement adjunct for diabetic ulcers.

In some embodiments, the therapeutic protein is useful in treating decubitus ulcer, varicose ulcer, debridement of eschar, dehiscent wound, sunburn, or acute decompensated congestive heart failure.

In some embodiments, the therapeutic protein is useful in enzymatic degradation of macromolecules. In some aspects of these embodiments, the therapeutic protein is useful in treating many types of dystonia (e.g., cervical), debridement of chronic dermal ulcers and severely burned areas, cystic fibrosis, respiratory tract infections, respiratory tract infections in selected patients with FVC greater than 40% of predicted, debridement of necrotic tissue, or debridement of necrotic tissue or liquefication of slough in acute and chronic lesions (e.g., pressure ulcers, varicose and diabetic ulcers, burns, postoperative wounds, pilonidal cyst wounds, carbuncles, and other wounds).

In some embodiments, the therapeutic protein is useful in treating cancer. In some aspects of these embodiments, the cancer is any one of cancers described herein.

In some embodiments, the therapeutic protein is useful in treating inflammatory disease or condition. In some aspects of these embodiments, the inflammatory disease or condition is any one diseases or conditions described herein (e.g. rheumatoid arthritis, Crohn's disease, psoriasis, and multiple sclerosis).

In some embodiments, the therapeutic protein is useful in organ transplantation (e.g. treating acute kidney transplant rejection).

In some embodiments, the therapeutic protein is useful in treating pulmonary disorders (e.g., respiratory syncytial virus infection, asthma).

In some embodiments, the therapeutic protein is useful in treating infectious disease. In some aspects of these embodiments, infectious disease is HIV infection.

Further examples of useful therapeutic proteins can be found in U.S. Pat. Nos. 8,349,910; and 8,043,833; U.S. patent applications 2013/0195888; and 2007/0092486; and PCT WO 2014/130064, each of which is hereby incorporated by reference in its entirety. In some embodiments, biomolecules can be sensitive to physiological environments, e.g., to physiologic enzymes or local pH, before delivery to the target tissue or target cell.

In some embodiments, the biomolecule is an antibody-drug conjugate. In some embodiments, the antibody-drug conjugate is trastuzumab-emtansine, brentuximab-vedotin, or T-DM1.

In some embodiments, the biomolecule is an antibody fragment-drug conjugates; protein-drug conjugates; peptide-drug conjugates (e.g., paclitaxel-Angiopep 2, BMTP-11 (Arrowhead Research), zoptarelin doxorubicin, and NGR-hTNF).

In some embodiments, the biomolecule is a fusion protein (i.e., a chimeric protein formed by the expression of two or more genes that encode for different proteins). In some embodiments the fusion protein is Fc fusion protein, which contain an antibody Fc unit that can offer stability or selective targeting of a cell or tissue type, including therapeutic proteins, such as atacicept, abatacept, aflibercept, alefacept, belatacept, etanercept, sotatercept, romiplostim, and rilonacept In some embodiments, the biomolecule is a bispecific fusion protein (i.e., bispecific antibodies), which comprise two arms from different antibodies, and are thereby able to target two different types of antigens, such as Ec-LDP-Hr-AE, MM-111 (Merrimack Pharmaceuticals), and IMCgp100 (Immunocore Ltd.).

In some embodiments, the biomolecule is a multimeric fusion protein, which is a fusion protein created by engineered multimerization (e.g., with streptavidin or using leucine zippers), such as polyvalent IgG2a Fc (M045).

Antigens and Adjuvants

In some embodiments of the present disclosure, the payload is an antigen.

In some embodiments, the antigen is hepatitis B surface antigen. In some embodiments, the antigen is strains 6, 11, 16, or 18 of HPV (Human papillomavirus). In some embodiments, the antigen is a capsid protein from HPV. In some embodiments, the antigen is OspA. In some embodiments, the antigen is lipoprotein on outer surface of Borrelia burgdorferi. In some embodiments, the antigen is Anti-Rhesus (Rh) immunoglobulin G. In some embodiments, the antigen is HIV antigen. In some embodiments, the antigen is hepatitis C antigen.

In some embodiments, the antigen is Influenza virus antigen. In some embodiments, the antigen is influenza B virus antigen. In some embodiments, the antigen is influenza A virus antigen.

In some embodiments, the antigen is poliovirus antigen.

In some embodiments, the antigen is a dust mite allergen. In some embodiments, the dust mite allergen is any one of dust mite allergens disclosed in, e.g., Stewart, Clinical Reviews in Allergy and Immunology 1995, 13, 135-150, the disclosure of which is incorporated herein by reference in its entirety.

In some embodiments, the antigen is a peptide. In some embodiments, the peptide is SIINFEKL (Ova257-264). In some embodiments, the antigen is palmitic acid conjugated to SIINFEKL peptide (C16-SIINFEKL).

In some embodiments of the present disclosure, when the payload is an antigen, the core of the particle optionally comprises an adjuvant as an additional payload. In some embodiments, adjuvant is as immunostimulatory agent. In some embodiments, the adjuvant is selected from the group consisting of resiquimod, imiquimod, gardiquimod, flagellin, monophosphoryl lipid A, N-glycolyted muramyldipeptide, CpG, R848 and Cholera toxin. In some embodiments, adjuvant is palmitic acid conjugated to R848 (C16-R848). In some embodiments, adjuvant is an inorganic compound. In some embodiments, adjuvant is alum, aluminum hydroxide, aluminum phosphate, or calcium phosphate hydroxide. In some embodiments, adjuvant is paraffin oil. In some embodiments, adjuvant is Bordetella pertussis, Mycobacterium bovis, or toxoids. In some embodiments, adjuvant is squalene, thimerosal, quil A, quillaja, soaybean, polygala senega, IL-1, IL-2, IL-12, Freund's complete adjuvant, Freund's incomplete adjuvant, Adjuvant 65. In some embodiments, adjuvant is a peanut oil based.

Polynucleotides and Nucleic Acids

In some embodiments, payload is a polynucleotide or a nucleic acid, such as, e.g., RNA (e.g., mRNA, microRNA, siRNA, or shRNA) or DNA (e.g., cDNA).

The nucleic acid may be double-stranded (e.g., double-stranded DNA) or single-stranded (e.g., single-stranded RNA). The nucleic acid can comprise a vector (e.g., a plasmid or a viral vector, e.g., one derived from a retrovirus, a lentivirus, an adenovirus, or an adeno-associated virus). In some embodiments, the nucleic acid can reduce expression of a protein (e.g., a protein associated with a disease state, e.g., a kinase upregulated in a cancer, such as BRAF-mutated melanoma). In some embodiments, the nucleic acid can introduce or enhance expression of a protein (e.g., to encode for a protein that is depleted in a disease state, e.g., normal CFTR protein to treat cystic fibrosis).

In some embodiments, the siRNA is siMYC (i.e., anti-MYC siRNA). In some embodiments, the siRNA is si-c-MYC (i.e., anti-c-MYC siRNA). In some embodiments, the siRNA is siBRAF (i.e., anti-BRAF siRNA). In some embodiments, the siRNA is siBRAF^{V600E} (i.e., anti-BRAF^{V600E} siRNA).

In some embodiments, polynucleotide (e.g., siRNA, miR, mRNA) may target the expression and/or activity of one or more proteins (e.g., an enzyme, e.g., a kinase) associated with cancer In some embodiments, the polynucleotide can target a protein selected from the group consisting of: kinesin spindle protein (KSP), RRM2, keratin 6a (K6a), HER1, ErbB2, a vascular endothelial growth factor (VEGF) (e.g., VEGFR1, VEGFR3), a platelet-derived growth factor receptor (PDGFR) (e.g., PDGFR-□, PDGFR-□), epidermal growth factor receptor (EGFR), a fibroblast growth factor receptor (FGFR) (e.g., FGFR1, FGFR2, FGFR3, FGFR4), EphA2, EphA3, EphA4, HER2, HER3, HER4, INS-R, IGF-1R, IR-R, CSF1R, KIT, FLK-II, KDR/FLK-1, FLK-4, flt-1, c-Met, Ron, Sea, TRKA, TRKB, TRKC, FLT3, VEGFR/F1t2, Flt4, EphA1, EphB2, EphB4, Piml, Pim2, Pim3, Tie2PKN3, PLK1, PLK2, PLK3, Src, Fyn, Lck, Fgr, Btk, Fak, SYK, FRK, JAK, Abl, Kit, KDR, CaM-kinase, phosphorylase kinase, MEKK, ERK, mitogen activated protein (MAP) kinase, phosphatidylinositol-3-kinase (PI3K), an AKT (e.g., Aktl, Akt2, Akt3), TGF-□R, KRAS, BRAF, a cyclin-dependent kinase (e.g., CDK1, CDK2, CDK4, CDK5, CDK6, CDK7, and CDK9), GSK3, a CDC-like kinase (CLK) (e.g., CLK1, CLK4), an Aurora kinase (e.g., Aurora A, Aurora B, and Aurora C), a mitogen-activated protein kinase kinase (MEK) (e.g., MEK1, MEK2), mTOR, protein kinase A (PKA), protein kinase C (PKC), protein kinase G (PKG), and PHB1.

In some embodiments, the biomolecule is mRNA encoding a cancer antigen, for example an mRNA encoding CA125 (ovarian cancer), CA 19-9 (pancreatic cancer), or CA27.29 (breast cancer) protein antigen.

Methods of Making

In some embodiments, the present disclosure provide a making a particle, the method comprising: i) combining a silica particle with a polyamine to obtain a polyamine-coated silica particle; ii) combining the polyamine-coated silica particle with a crosslinker to obtain a crosslinked polyamine-coated silica particle; iii) combining the crosslinked polyamine-coated silica particle with a polysaccharide to obtain a polysaccharide-coated silica particle; and iv) combining the polysaccharide-coated silica particle with a fluoride source to remove the silica particle to obtain the particle.

In some embodiments, the present disclosure provides a method of making a particle, the method comprising: i) combining a silica particle with a polyamine to obtain a polyamine-coated silica particle; ii) combining the polyamine-coated silica particle with a crosslinker to obtain a crosslinked polyamine-coated silica particle; iii) combining the crosslinked polyamine-coated silica particle with a biomolecule to obtain a biomolecule-containing silica particle; iv) combining the biomolecule-containing silica particle with a polysaccharide to obtain a polysaccharide-coated silica particle; and iv) combining the polysaccharide-coated silica particle with a fluoride source to remove the silica particle to obtain the particle.

In these embodiments, the polyamine, the crosslinker, the biomolecule, and the polysaccharide are as described herein. In some embodiments, the silica particle is a nanoparticle with an average diameter of about 200-400 nm, or about 200-300 nm. In some embodiments, the silica is carboxylated. For example, the silica has formula SiO₂—(CH₂)ₙ—COOH, wherein n is an integer from 0 to 10. In some embodiments, the silica comprises from about 1 wt. % to about 35 wt % of COOH groups, or about 1 wt. %, about 2 wt. %, about 2.5 wt %, or about 5 wt. % of COOH groups.

Without being bound by any theory, it is believed that the fluoride source diffuses through the polysaccharide coating and the polyamine layer, reacts with the silica and dissolves the silica. The resultant water-soluble silicon fluoride diffuses back into solution, thereby leaving a hollow core of the particle. In some embodiments the fluoride source is a fluoride salt, such as NaF, KF, NH₄F, or a complex containing a F⁻, where F⁻ is attached to its counteraction through ionic bonds, electrostatic interactions, or Van Der Waals bonds.

Any of the steps of any of the methods of making the particle described herein may be performed in water, dextrose solution, saline, buffer, for example, at room temperature, with or without agitation. For coating the silica particle with polyamine and polysaccharide, a layer-by-layer coating technique may be employed or any other suitable technique.

In some embodiments, the present disclosure provides a particle prepared by any one of the methods describe herein. For example, the disclosure provides a particle comprising a shell comprising a crosslinked polyamine layer with a polysaccharide coating, prepared by any one of the methods described herein. In another example, the disclosure provides a particle comprising a shell comprising a crosslinked polyamine layer with a polysaccharide coating, and a biomolecule between the crosslinked polyamine layer and the polysaccharide coating, prepared by any one of the methods described herein.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2nd Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6th Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4th Ed., Wiley & Sons, Inc., New York (2006).

Methods of Use

Polysaccharide-coated particles (e.g., nanocapsules) of the present disclosure, upon contact with the antigen-presenting cell and upon binding to a pattern recognition receptor (PRR), activate molecular pathways leading to immunogenic response by the antigen-presenting cell. In some embodiments, the PRR is selected from the group consisting of: a membrane-bound receptor kinase, a toll-like receptor, a C-type lectin receptor, a mannose receptor, asialoglycoprotein receptor, NOD-like receptor, and RIG-I-like receptor. Suitable examples of PRR include Dectin-1 receptor, Dectin-2 receptor, CD206 receptor, CD209 (DC-SIGN) receptor, Mincle receptor, TLR-2 and TLR-4 receptors. The immunogenic responses include: (i) inductions and release of chemoattractants, such as IP-10, MCP-1a, and MCP-3 (e.g., in locally treated tumors), (ii) recruitment of immune cells near the tumor cites, (iii) promoting secretion of an inflammatory cytokine (e.g., IL-1, IL-12, IL-18, TNF-α, INF-γ), modulating a lymph node drainage, and/or promoting expression of a disease-specific antigen by the antigen-presenting cell. When the particle of the present disclosure comprises a nucleic acid encoding a pathogen/disease-specific antigen, the antigen presenting cell transfects and expresses the protein antigen, thereby starting the cascade of creating antibodies against that specific antigen (and the pathogen/disease comprising the antigen) by the cells of the immune system. The particles are useful, therefore, for vaccinating the subject against the pathogens containing the antigen. For example, if the antigen protein is a cancer-specific antigen, the particles are useful as a vaccine against cancer. Alternatively, the particle may comprise a protein that is a pathogen/disease-specific antigen. In this case, the APS need not transfect and/or express the protein. The APS initiates the molecular mechanisms to present the antigen to the immune system cells for the antibody generation.

Vaccination

Vaccination with messenger RNA (mRNA) is a complementary approach to traditional subunit vaccines based on peptides and proteins. The advantage of mRNA vaccines include the possibility of encoding a wide range of protein antigens, efficient manufacturing process and excellent safety profiles that enable multiple administrations. In addition, mRNA vaccines can be designed to encode multiple epitopes in a single mRNA molecule, and the inherent immunogenic property of RNA can be exploited for immune stimulation, thus facilitating vaccine design and development. The particles of the present disclosure advantageously overcome the shortcomings of the traditional mRNA vaccine approach, such as rapid enzymatic degradation and poor pharmacokinetics. In the particles of the present application, mRNA vaccines encoding disease-specific antigens are advantageously shielded from enzymatic degradation and show excellent pharmacokinetics and pharmacodynamics. When the disease against which the subject is been vaccinated is cancer, the protein antigen encoded by mRNA may be CA125 (ovarian cancer), CA 19-9 (pancreatic cancer), or CA27.29 (breast cancer). In the alternative, the cancer antigen may be a cancer protein obtained from the subject having the particular cancer. The cancer antigens may be obtained from the subjects surgically or laparoscopically, and further purified for encapsulation in the particles of the present disclosure.

Adjuvant Therapy

When the particle of the present disclosure does not contain a biomolecule (e.g., when the particle comprises a hollow core, a layer of crosslinked polyamine, and a layer of polysaccharide having the pathogen-associated molecular pattern), the particle, upon contact with the APC (in vivo or in vitro) triggers the secretion of inflammatory cytokines and coordinates the induction of adaptive immune responses against a disease or a pathogen. In one example, upon local (e.g., intra-tumor) administration of the particle to the patient, the particle induces an inflammatory response by the innate immune cells, such as DCs and macrophages. The particles, therefore, can substantially potentiate the cancer therapy. Not only the particles generate immune response against the tumor, but also induce significant lymph node drainage, further improving the outcome of the treatment.

Antigen Presenting Cells

In some embodiments, the antigen presenting cells capable of being activated by the particles of the present disclosure are selected from the group consisting of dendritic cells (e.g., CDS6), macrophages (e.g., CDS0), and natural killer cells (e.g., CD107a). Natural killer cells (also known as NK cells, K cells, and killer cells) are a type of lymphocyte (a white blood cell) and a component of innate immune system. NK cells play a major role in the host-rejection of both tumors and virally infected cells. Dendritic cells (DCs) are key professional antigen presenting cells (APCs) that phagocytose, process and present antigens to T-cells (e.g., in lymph nodes) for the initiation of antigen-specific immune responses. DCs recognize the conserved microbial molecular structures of pathogen, termed pathogen-associated molecular patterns (PAMPs), via the engagement of the pattern recognition receptors (PRRs). The PRRs include, for example, Dectin-1, Dectin-2, CD206, CD209 (DC-SIGN), Mincle, TLR-2 and TLR-4 receptors.

Cancer

The particles and biomolecules disclosed herein are useful for modulating (e.g., stimulating or enhancing) an immune response, and therefore a useful for treating cancer, for example, by administering to a subject an amount of the particle or the biomolecule that is effective to stimulate T cells in the subject having cancer. Exemplary types of cancer that may be treated with the provided compounds include: bladder, brain, breast, cervical, colorectal, esophageal, kidney, liver, lung, nasopharyngeal, oral and oropharyngeal, pancreatic, prostate, skin, stomach, uterine, ovarian, thyroid, testicular, tumors with fibrotic stroma, uterine and hematologic cancer.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

In some embodiments, the cancer is selected from the group consisting of: a kidney cancer, a liver cancer, a breast cancer, a lung cancer, a pancreatic cancer, a bladder cancer, a colon cancer, a melanoma, a thyroid cancer, an ovarian cancer, and a prostate cancer. The cancer may be, for example, any one of the following cancers:

i) breast cancers, including, for example ER+ breast cancer, ER− breast cancer, her2− breast cancer, her2+ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER−), progesterone receptor negative, and her2 negative (her2-). In some embodiments, the breast cancer may have a high risk Oncotype score;

ii) hematopoietic cancers, including, for example, leukemia (acute lymphocytic leukemia (ALL), acute myelogenous leukemia (AML), chronic lymphocytic leukemia (CLL), chronic myelogenous leukemia (CIVIL), hairy cell leukemia), mature B cell neoplasms (small lymphocytic lymphoma, B cell prolymphocytic leukemia, lymphoplasmacytic lymphoma (such as Waldenstrom's macroglobulinemia), splenic marginal zone lymphoma, plasma cell myeloma, plasmacytoma, monoclonal immunoglobulin deposition diseases, heavy chain diseases, extranodal marginal zone B cell lymphoma (MALT lymphoma), nodal marginal zone B cell lymphoma (NMZL), follicular lymphoma, mantle cell lymphoma, diffuse B cell lymphoma, diffuse large B cell lymphoma (DLBCL), mediastinal (thymic) large B cell lymphoma, intravascular large B cell lymphoma, primary effusion lymphoma and Burkitt lymphoma/leukemia), mature T cell and natural killer (NK) cell neoplasms (T cell prolymphocytic leukemia, T cell large granular lymphocytic leukemia, aggressive NK cell leukemia, adult T cell leukemia/lymphoma, extranodal NK/T cell lymphoma, enteropathy-type T cell lymphoma, hepatosplenic T cell lymphoma, blastic NK cell lymphoma, mycosis fungoides (Sézary syndrome), primary cutaneous anaplastic large cell lymphoma, lymphomatoid papulosis, angioimmunoblastic T cell lymphoma, unspecified peripheral T cell lymphoma and anaplastic large cell lymphoma), Hodgkin lymphoma (nodular sclerosis, mixed celluarity, lymphocyte-rich, lymphocyte depleted or not depleted, nodular lymphocyte-predominant), myeloma (multiple myeloma, indolent myeloma, smoldering myeloma), chronic myeloproliferative disease, myelodysplastic/myeloproliferative disease, myelodysplastic syndromes, immunodeficiency-associated lymphoproliferative disorders, histiocytic and dendritic cell neoplasms, mastocytosis, chondrosarcoma, Ewing sarcoma, fibrosarcoma, malignant giant cell tumor, and myeloma bone disease;

iii) lung cancers, including, for example, bronchogenic carcinoma, e.g., squamous cell, undifferentiated small cell, non-small cell lung cancer (NSCLC), undifferentiated large cell, and adenocarcinoma; alveolar and bronchiolar carcinoma; bronchial adenoma; sarcoma; lymphoma; chondromatous hamartoma; and mesothelioma;

iv) genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;

v) liver cancers, including, for example, hepatoma, e.g., hepatocellular carcinoma; cholangiocarcinoma; hepatoblastoma; angiosarcoma; hepatocellular adenoma; hepatobiliary carcinoma (HCC), and hemangioma;

vi) kidney (renal) cancers, including, for example, clear cell renal cell carcinoma (ccRCC), papillary renal cell carcinoma, chromophobe renal cell carcinoma, collecting duct renal cell carcinoma, unclassified renal cell carcinoma, transitional cell carcinoma, and renal sarcoma;

vii) bladder cancers, including, for example, transitional cell carcinoma, urothelial carcinoma, papillary carcinoma, flat carcinoma, squamous cell carcinoma, adenocarcinoma, small-cell carcinoma, and sarcoma;

viii) gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, epithelial cancer, mucinous cystadenocarcinoma, unclassified carcinoma, granulosa thecal cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma;

ix) skin cancers, including, for example, cutaneous melanoma, malignant melanoma, basal cell carcinoma, squamous cell carcinoma, Kaposi's sarcoma, moles dysplastic nevi, lipoma, angioma, dermatofibroma, keloids, psoriasis;

x) thyroid cancers, including, for example, papillary thyroid cancer, follicular thyroid cancer, anaplastic thyroid carcinoma, and medullary thyroid cancer; and

xi) adrenal gland cancers, including, for example, neuroblastoma.

In some embodiments, the cancer is a solid tumor. In some embodiments, the cancer is a fibrotic tumor. In some embodiments, the cancer is colon carcinoma. In some embodiments, the cancer is melanoma.

Combination Therapies

The particles and compositions of the present disclosure can be administered to a subject (e.g., in need of the administration of the particle) alone or in combination with one or more additional therapeutic agents, or a pharmaceutically acceptable salt thereof.

The additional therapeutic agents are selected based on the condition, disorder or disease to be treated. For example, a particle can be co-administered with one or more additional agents that function to enhance or promote an immune response in a subject.

In some embodiments, the particle of the present disclosure, or a composition comprising same, can be administered in combination with and adjuvant (e.g., immunoadjuvant). In some embodiments, adjuvant is as immunostimulatory agent. In some embodiments, the adjuvant is selected from the group consisting of resiquimod, imiquimod, gardiquimod, flagellin, monophosphoryl lipid A, N-glycolyted muramyldipeptide, CpG, R848 and Cholera toxin. In some embodiments, adjuvant is palmitic acid conjugated to R848 (C16-R848). In some embodiments, adjuvant is an inorganic compound. In some embodiments, adjuvant is alum, aluminum hydroxide, aluminum phosphate, or calcium phosphate hydroxide. In some embodiments, adjuvant is paraffin oil. In some embodiments, adjuvant is Bordetella pertussis, Mycobacterium bovis, or toxoids. In some embodiments, adjuvant is squalene, thimerosal, quil A, quillaja, soaybean, polygala senega, IL-1, IL-2, IL-12, Freund's complete adjuvant, Freund's incomplete adjuvant, Adjuvant 65. In some embodiments, adjuvant is a peanut oil based.

In some embodiments, the particle of the present disclosure, or a composition comprising same, can be administered to the subject in combination with any one of the therapeutic biomolecules disclosed herein, or a pharmaceutically acceptable salt thereof, or a pharmaceutical composition comprising same.

In some embodiments, the additional therapeutic agent is cyclophosphamide. Cyclophosphamide (CPA, Cytoxan, or Neosar) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety). Ifosfamide (MITOXANAO) is a structural analog of cyclophosphamide and its mechanism of action is considered to be identical or substantially similar to that of cyclophosphamide.

In some embodiments, the additional therapeutic agent is an anticancer agent (e.g., paclitaxel, protein-bound paclitaxel, docetaxel, doxorubicin, pegylated liposomal doxorubicin, daunorubicin, epirubicin, eribulin, fluorouracil, gemcitabine, ixabelipone, melphalan, cis-platin, carboplatin, cyclophosphamide, mitomycin, methotrexate, mitoxantrone, vinolrebine, vinblastine, vincristine, ifosfamide, teniposide, etoposide, bleomycin, leucovorin, tamoxifen, taxol, trifluridine, herceptin, avastin, cytarabine, dactinomycin, interferon alpha, streptozocin, prednisolone, irinotecan, sulindac, 5-fluorouracil, capecitabine, oxaliplatin/5 FU, abiraterone, letrozole, 5-aza/romidepsin, or procarbazine, or a pharmaceutically acceptable salt thereof). In certain embodiments, the anticancer agent is paclitaxel or docetaxel, or a pharmaceutically acceptable salt thereof.

In some embodiments, the additional therapeutic agent is a targeted therapy. In some embodiments, the targeted therapy is selected from the group consisting of: bevacizumab, regorafenib, aflibercept, ramucirumab, a HER2 targeted agent (e.g. trastuzumab, pertuzumab, neratinib), a BRAF targeted agent (e.g. dabrafenib, encorafenib, vemurafenib), a MEK targeted agent (e.g. trametinib, cobimetinib), an epidermal growth factor receptor (EGFR) targeted agent (e.g. cetuximab, panitumumab), a KIT targeted agent (e.g. dasatinib, imatinib, nilotinib).

In some embodiments, the additional therapeutic agent is a checkpoint inhibitor. In some embodiments, the checkpoint inhibitor is selected from the group consisting of: a programmed cell death protein-1 (PD-1) inhibitor, an inhibitor of programmed death-ligand-1 (PD-L1) or programmed death-ligand-2 (PD-L2), an inhibitor of cytotoxic T-lymphocyte-associated protein-4 (CTLA-4), an inhibitor of Lymphocyte-activation gene 3 (LAG-3), an inhibitor of Cluster of Differentiation 47 (CD47), an inhibitor of Signal regulatory protein α (SIRP α) (e.g., TTI-621, OSE-172), an inhibitor of T-cell immunoglobulin and mucin-domain containing-3 (TIM-3), an inhibitor of V-domain Ig suppressor of T cell activation (VISTA), an inhibitor of T cell immune response cDNA 7 (TIRC7) protein and an inhibitor of indoleamine (2,3)-dioxygenase-1 (IDO1), inihibitor of indoleamine (2,3)-dioxygenase-2 (IDO2). In some embodiments, the checkpoint inhibitor is a dual inhibitor of LAG-3 and PD-1 (e.g., MGD013). In some embodiments, the checkpoint inhibitor is selected form the group consisting of: an anti-PD-1 antibody (e.g., pembrolizumab, nivolumab), an anti-PD-L1 antibody (e.g., atezolizumab), an anti-PD-L2 antibody, an anti-CTLA-4 antibody (e.g., ipilimumab), and an anti-TIM-3 antibody (e.g., TSR-022). In some embodiments, the checkpoint inhibitor is selected form the group consisting of: PD-1 inhibitor, PD-L1 inhibitor, PD-L2 inhibitor, TIM-1 inhibitor, TIM-3 inhibitor, LAG-3 inhibitor, CTLA-4 inhibitor, CD-47 inhibitor, SIRPa inhibitor, and VISTA inhibitor. In some embodiments, the checkpoint inhibitor is selected form the group consisting of: durvalumab, pembrolizumab, nivolumab, atezolizumab, avelumab, ipilimumab, and TSR-022. In some embodiments, the checkpoint inhibitor is selected form the group consisting of: avelumab, durvalumab, pembrolizumab, nivolumab, atezolizumab, ipilimumab, TSR-022, MGD013, TTI-621, OSE-172 and CA-170. In some embodiments, the method comprises administering to the subject at least two of: avelumab, durvalumab, pembrolizumab, nivolumab, atezolizumab, ipilimumab, and TSR-022. In some embodiments, the method comprises administering to the subject at least two of: avelumab, durvalumab, pembrolizumab, nivolumab, atezolizumab, ipilimumab, TSR-022, MGD013, TTI-621, OSE-172 and CA-170. In some embodiments, the method comprises administering to the subject at least three of: avelumab, durvalumab, pembrolizumab, nivolumab, atezolizumab, ipilimumab, and TSR-022. In some embodiments, the method comprises administering to the subject at least three of: avelumab, durvalumab, pembrolizumab, nivolumab, atezolizumab, ipilimumab, TSR-022, MGD013, TTI-621, OSE-172 and CA-170. In some embodiments, the method comprises administering to the subject pembrolizumab and nivolumab. In some embodiments, the method comprises administering to the subject avelumab, durvalumab, pembrolizumab, nivolumab, atezolizumab, and ipilimumab. In some embodiments, the method comprises administering to the subject avelumab, durvalumab, pembrolizumab, nivolumab, atezolizumab, ipilimumab, and TSR-022. In some embodiments, the method comprises administering to the subject avelumab, durvalumab, pembrolizumab, nivolumab, atezolizumab, ipilimumab, TSR-022, MGD-013, TTI-621, OSE-172 and CA-170. In some embodiments, the checkpoint inhibitor is an antibody and is administered to the subject intravenously. In some embodiments, the checkpoint inhibitor is an antibody and is administered to the subject orally. In some embodiments, the checkpoint inhibitor is a small-molecule drug that is administered to the subject orally (e.g., CA-170). In some embodiments, when at least two checkpoint inhibitors are administered to the subject, at least one checkpoint inhibitor is administered orally, and at least one checkpoint inhibitor is administered intravenously. In some embodiments, all checkpoint inhibitors are administered intravenously.

In some embodiments, the checkpoint inhibitor is any one of checkpoint inhibitors described in Petrova et al., TTI-621 (SIRPaFc): A CD47-Blocking Innate Immune Checkpoint Inhibitor with Broad Anti-Tumor Activity and Minimal Erythrocyte Binding, Clinical Cancer Research, 2016 (DOI: 10.1158/1078-0432.CCR-16-1700). In some embodiments, the checkpoint inhibitor is inhibitor of CD47 (receptor) or SIRPalpha (ligand). In some embodiments, the checkpoint inhibitor is TTI-621, that binds to and neutralizes CD47, produced by Trillium Therapeutics Inc; or OSE-172, antagonist of SIRPα, produced by OSE Immunotherapeutics. In some embodiments, the checkpoint inhibitor is an inhibitor of LAG-3 (CD223) (e.g., MGD013, a dual inhibitor of LAG-3 and PD-1, manufactured by MacroGenics). In some embodiments, the checkpoint inhibitor is V-domain Immunoglobulin Suppressor of T-cell Activation (VISTA) antagonist (e.g., CA-170 manufactured by Curtis Inc). In some embodiments, the checkpoint inhibitor selectively targets and inhibit both PD-L1 and VISTA (e.g., CA-170). In some embodiments, the checkpoint inhibitor is PD-L1/VISTA antagonist. In some embodiments, the checkpoint inhibitor is an inhibitor of IDO1 (e.g. BMS-986205 developed by Flexus Inc., and Bristol-Myers Squibb, epacadostat, indoximod, navoximod, NLG802, HTI-1090).

Salts and Pharmaceutically Acceptable Salts

In some embodiments, a salt of any one of the compound disclosed herein is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of any one of the compounds disclosed herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form salts (e.g., pharmaceutically acceptable salts) of any one of the compounds disclosed herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Pharmaceutical Compositions

The present application also provides pharmaceutical compositions comprising a particle disclosed herein, or a pharmaceutically acceptable salt thereof and a pharmaceutically acceptable carrier.

The pharmaceutical composition may also comprise any one of the additional therapeutic agents described herein (e.g., cancer antigens, cancer-specific antibodies, such as checkpoint inhibitors, or any other agents disclosed in this application)

In certain embodiments, the application also provides pharmaceutical compositions and dosage forms comprising any one the additional therapeutic agents described herein, or a pharmaceutically acceptable salt thereof.

The carrier(s) are “acceptable” in the sense of being compatible with the other ingredients of the formulation and, in the case of a pharmaceutically acceptable carrier, not deleterious to the recipient thereof in an amount used in the medicament. Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%400% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in exosomes, liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a particle disclosed herein is present in an effective amount (e.g., a therapeutically effective amount). In one example, when the particle comprises a biomolecule, the effective amount of the particle comprises and effective amount of the biomolecule.

Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound of any one of the Formulae disclosed herein, or a pharmaceutically acceptable salt thereof, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a particle is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent, such as a PD-1 inhibitor, in any one of amounts and dosage forms described herein.

### Definitions

Unless defined otherwise, all technical and scientific terms used herein have the same meaning as is commonly understood by one of ordinary skill in the art to which this disclosure belongs.

As used herein, the term “about” means “approximately” (e.g., plus or minus approximately 10% of the indicated value).

As used herein, the singular forms “a,” “an,” and “the” include plural referents unless the context clearly dictates otherwise.

For the terms “e.g.” and “such as,” and grammatical equivalents thereof, the phrase “and without limitation” is understood to follow unless explicitly stated otherwise.

The term “particle” as used herein refers to a composition having a size from about 1 nm to about 1000 □m.

The term “microparticle” as used herein refers to a particle having a size from about 1000 nm to about 1 mm.

The term “nanoparticle” as used herein refers to a particle having a size from about 1 nm to about 1000 nm.

The term “particle size” (or “nanoparticle size” or “microparticle size”) as used herein refers to the median size in a distribution of nanoparticles or microparticles. The median size is determined from the average linear dimension of individual nanoparticles, for example, the diameter of a spherical nanoparticle. Size may be determined by any number of methods in the art, including dynamic light scattering (DLS) and transmission electron microscopy (TEM) techniques.

The term “Encapsulation efficiency” (EE) as used herein is the ratio of the amount of drug that is encapsulated by the particles (e.g., nanoparticles) to the initial amount of drug used in preparation of the particle.

The term “Loading capacity” (LC) or “loading efficiency” (LE) as used herein is the mass fraction of drug that is encapsulated to the total mass of the particles (e.g., nanoparticles).

“Polymer” as used herein, is given its ordinary meaning as used in the art, i.e., a molecular structure including one or more repeat units (monomers), connected by covalent bonds.

“Copolymer” as used herein refers to a polymer with more than one type of repeat unit present within the polymer.

As used herein, the term “adjuvant” refers to an immunological adjuvant. By this is meant a compound or composition that is able to enhance or facilitate the immune system's response to a pathogen, thereby inducing an immune response or series of immune responses in the subject. The adjuvant can facilitate the effect of the compositions, e.g., by forming depots (prolonging the half-life of the composition), provide additional T-cell help, and/or stimulate cytokine production.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the pattern recognition receptor (or the antigen-presenting cell) with a particle of this disclosure includes the administration of the particle to an individual or patient, such as a human, having the pattern recognition receptor (or the antigen-presenting cell), as well as, for example, introducing the particle into a sample containing a cellular or purified preparation containing the pattern recognition receptor (or the antigen-presenting cell).

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans. In some embodiments, the subject demonstrates resistance to immunotherapies.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### Examples

Description of Experiments

Reagents and Instruments.

Experimental Details. Carboxyl-functionalized silica nanoparticles (about 237 nm in diameter) was purchased from microparticles GmbH (Volmerstraβe 9, 12489 Berlin, Germany). Sodium meta-periodate and dimethyl 3,3′-dithiobispropionimidate×2HCl (DTBP):

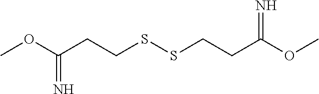

were obtained from Thermofisher Scientific (Rockford, Ill. U.S.A.). Mannan from Saccharomyces cerevisiae, triethylamine (TEA), ammonium fluoride and polyethyleneimine (PEI) (branched, MW 25,000) were obtained from Sigma-Aldrich (St. Louis, Mo. USA). mRNA encoding EGFP and OVA protein were from Trilink (San Diego, Calif., USA). UV-Vis absorption and fluorescence were measured using BioTek synergy neo microplate reader. Transmission electron microscope (TEM) images were acquired using JEOL 1400-plus, and atomic force microscope (AFM) was performed using Asylum-1 MFP-3D. Hydrodynamic size and (zeta)-potential were measured using NanoSight NS300 and Malvern Zetasizer Nano ZSP. Flow cytometry analyses were performed using Ze5 (Beckman Coulter, USA) and the data were analyzed using FlowJo 10.2 software. The mRNA loading amount was analyzed using gel permeation chromatography (GPC, Shimadzu). Confocal microscope images were taken with Leica SP8 confocal microscope.

Oxidation of Polysaccharide (Polysaccharide-CHO).

Dextran (Dex) or mannan (Mann) was oxidized to generate aldehyde functional groups for further chemical modification. 0.2 mg of polysaccharide was dissolved in 5 mL of ultrapure water and mixed with 5 mL of 0.01 M sodium periodate solution, incubated for 1 h with gentle shaking at room temperature in the dark. The reactants were purified using dialysis membrane (MWCO=3,000 Da, Spectrum™) against deionized water for three days and lyophilized by freeze-drying in the dark for two to three days. The aldehyde content of polysaccharide-CHO was quantified using a modified hydroxylamine hydrochloride method. The resulting polysaccharide-CHO was stored at 4° C. in the dark until further use.

Synthesis and Characterization of Polysaccharide-Based Nanocapsule.

Carboxylated silica nanoparticles (siNPs) (about 200 nm in diameter) was used as a template to construct hollow polysaccharide nanocapsule. 150 μl of aqueous PEI25K solution (10 mg/mL in ultrapure water) was added to 15 mg of carboxylated siNP in ultrapure water (900 μl), followed by vigorous vortex for 10 min to introduce positive charge on the surface of carboxylated siNP (PEI-siNP). PEI-siNP was purified three times using ultra-centrifugation for 2 min at 18,500 rpm. PEI-siNP was then chemically crosslinked with dimethyl 3,3′-dithiobispropionimidate×2HCl (DTBP) crosslinker (0.5 mg in 1 mL 0.1 M TEA buffer at pH 8) for 1 h at room temperature followed by three rounds of purifications with ultrapure water using ultracentrifugation (2 min, at 18,500 rpm 4° C.). The PEI content was analyzed using 2,4,6-trinitrobenzene sulfonic acid (TNBSA, ThermoFisher) solution by quantifying primary amine groups per PEI. For mRNA loading, mRNA (100 μl, 500 μg/mL) was added to crosslinked-PEI-siNP solution (900 μl, 15 mg), incubated for 10 min with vigorous vortex and purified twice with ultrapure water. The mRNA loading was analyzed by agarose gel electrophoresis (15%, 100V, 20 min) and GPC equipped with TSKgel G3000SWxl column (7.8 mm ID 9 300 mm, Tosoh Bioscience LLC). Polysaccharide-CHO (1000 μL, 2 mg/mL) was chemically introduced into the outermost surface of PEI-siNPs in ultrapure water for 12 h at room temperature by amine-aldehyde reaction between PEI and polysaccharide-CHO (polysaccharide-siNP) (e.g., the surface of PEI-si-NP was covered by MANN-CHO by the chemical bonding of amine-PEI and CHO-MANN in DI water overnight followed by 3-times purification with DI water (MANN-si-NP)). For fluorescence labelling, amine-functionalized Cy5.5 (2 μl stock in DMSO) was added to the mixture of the PEI-si-NP and CHO-MANN solution. The siNP core of polysaccharide-siNP was removed by ammonium fluoride (300 μl) for 5 min at room temperature and washed three times with ultrapure water, followed by PBS twice to produce hollow polysaccharide-nanocapsule (e.g., MANN-NC). For physicochemical characterizations of hollow nanocapsule, TEM and AFM were performed. Nanocapsules were stored at 4° C. until further use. The endotoxin level for each in vivo dose of nanocapsule and polysaccharide were measured to be less than 0.05 EU/dose, as determined by the LAL Chromogenic Endotoxin Quantitation Kit (Pierce).

BMDC Uptake, Cytotoxicity and mRNA Transfection.

Bone marrow derived dendritic cells (BMDCs) were prepared according to the literature protocol (See Ref. 2). Briefly, cells were aseptically isolated from femurs and tibia of C57BL/6 mice and cultured in complete DC media comprised of RPMI-1640 (Gibco) supplemented with 10% fetal bovine serum (FBS, Corning), 1% penicillin/streptomycin (Gibco), 55 μM β-mercaptoethanol (Gibco), 20 ng/ml granulocyte-macrophage colony-stimulating factor (GM-CSF, Genscript) at 37° C. with 5% CO₂. Fresh media was added on day 3, and all media was renewed on day 6 and 8. The cultured BMDCs were used between days 7 and 12.

For NP dose- and time-dependent uptake study, immature BMDCs in complete DC media were plated and cultured overnight at 10⁵ cells per well in 96-well plate and treated with the varying concentrations of formulations for indicated duration. BMDCs were then harvested with cell dissociation reagent (StemPro Accutase, Gibco) and washed twice with cold PBS. BMDCs were incubated with anti-CD16/32 Fcγ blocking antibody (eBioscience) for 10 min and stained with anti-CD11c antibody (Biolegend) for 30 min at room temperature. The cells were washed twice with FACS buffer (1% BSA in PBS) and dispersed in FACS buffer with DAPI (300 nM) for flow cytometry analysis. For cytotoxicity study, after 24 h incubation with NPs, viable BMDCs were quantified by cell counting kit (CCK)-8 (Dojindo Molecular Technologies) according to the manufacturer's instructions. For mRNA transfection study, 5-methoxyuridine modified mRNA encoding EGFP (EGFP-mRNA) was loaded into nanocapsule as well as Lipofectamine® 2000 (ThermoFisher Scientific) and BPEI as control groups. All other procedures are same with the uptake study. For intracellular visualization of mRNA translation using confocal microscope, 5×10⁵ BMDCs seeded on coverslip-bottom 12-well plate were treated with Cy5.5 labeled nanocapsule loaded with EGFP-mRNA for 12 h. BMDCs were washed three times with cold PBS, stained with DAPI in PBS (300 nM) and fixed with 4% formaldehyde for 30 min. After mounting coverslips with a mounting media (Vector Laboratories), cells were imaged by a confocal microscope.

BMDC Activation and Antigen Presentation and Cross-Priming.

BMDCs in complete DC media were plated at 10⁵ cells per well in 96-well plate. After overnight incubation, BMDCs were treated with various NP formulations or control groups, including soluble OVA-mRNA (1 μg/well) and CpG (1 μM, Integrated DNA technologies). Supernatants were collected and analyzed for cytokines, including IL-12p′70, TNF-α and IL-6. BMDCs were then harvested and incubated with anti-CD16/32 blocking antibody at room temperature for 10 min, stained at room temperature for 30 min with fluorophore-labeled antibodies against CD11c (Biolegend), CD40 (e-bioscience), CD86 (eBioscience) and MHCII (Biolegend) or anti-mouse SIINFEKL/H-2Kb monoclonal antibody 25-D1.16 (eBioscience) for antigen presentation. Cells were analyzed by flow cytometry analysis.

T Cell Expansion.

To assess cross-priming of T cells by OVA-mRNA loaded nanocapsule (mOVA-nanocapsule), 10⁵ BMDCs in 96-well plates were pulsed with mOVA-nanocapsule or control groups, including OVA protein (250 μg/mL)/CpG (5 μg/mL) and soluble mOVA (5 μg/mL) for 4 or 24 h. BMDCs were washed three times with PBS to remove free NPs. Spleens were harvested from OT-I and OT-II transgenic mice and CD8α+ and CD4+ T-cells were isolated using a negative selection kit (Stemcell Technology), respectively. After labeling them with carboxyfluorescein succinimidyl ester (CF SE, 0.1 μM, 2×106 cells/mL, 10 min in RPMI), T-cells were washed and transferred to the BMDC-containing 96-wells. After three days of co-culture, T-cells were harvested and stained with anti-CD8a (eBioscience) for flow cytometry analysis.

Pattern Recognition Receptor Inhibition Study with Blocking Antibodies (ELISA and Multiplex).

2×10⁵ BMDCs in complete DC media were seeded on 24-well plate overnight. Each PRR receptor on BMDCs surface was blocked by the corresponding blocking antibody for 30 min at final concentration of 2 μg/mL against Dectin-1, Dectin-2, CD206, CD209 (DC-SIGN), Mincle, TLR-2 and TLR-4 receptors, followed by NP treatment. All PRR blocking antibodies were from Invivogen. Phagocytosis inhibitor, latrunculin (Invitrogen), was also included for some groups. After 24 h incubation, supernatants were collected for cytokine analysis by ELISA (R&D system) or luminex multiflex assays (Luminex MAGPIX®).

Lymphatic draining efficacy. C57BL/6 mice were subcutaneously administered at the tail base with native-Mann, silica-Mann and capsule-Mann. The Cy5.5 fluorescence intensity of the three formulations were quantified and normalized before injection using a microplate reader with Eₓ=650 nm and Eₘ=665 nm. Inguinal LNs were harvested and imaged using IVIS to quantify Cy5.5 fluorescence signals. Then, LNs were homogenized by pestle motor (Argos Technologies) and treated with collagenase type IV (1 mg/mL) and DNase I (100 U/mL) at 37° C. for 30 min with gentle shaking to prepare single cell suspensions. The LN cells were filtered through a 40 μm cell strainer, washed twice and stained by fluorophore labeled antibodies for flow cytometry analysis. In detail, DCs were identified by antibodies against MHCII (Biolegend) and CD11c (Biolegend), and their activation was examined by antibodies against CD86 (BD Biosciences) and CD80 (BD Biosciences).

Animal Experiments.

Animals were cared for following the federal, state, and local guidelines. All work performed on animals was in accordance with and approved by University Committee on Use and Care of Animals (UCUCA) at University of Michigan, Ann Arbor. Female C57BL/6 (5-6 weeks) were purchased from Envigo (USA). For mRNA vaccination study, C57BL/6 mice were subcutaneously injected with 2×10⁵ B16F100VA cells per mouse at the right flank on day 0, and immunized on days 7, 12 and 17 with mOVA (10 μg), mOVA-Dex-capsule, mOVA-Mann-capsule (10 μg mOVA). Tumor growth was measured two to three times a week, and the tumor volume was calculated by the ellipsoidal calculation as V=(width)²×length×0.52. Animals were euthanized, when individual tumor masses reached 1.5 cm in diameter or when animals became moribund.

Tetramer Staining and ELISPOT.

For analysis of tumor antigen-specific CD8α+ T cells in the systemic circulation, submandibular bleeding was performed at indicated time points. Red blood cells in PBMCs were lysed with ACK lysis buffer twice at room temperature, followed by two washing steps with FACS buffer. PBMCs were blocked with CD16/32 antibody for 10 min and then stained with H-2Kb OVA tetramer-SINNFEKL (MBL International) and anti-CD8a (BD Biosciences) for flow cytometry analysis. ELISPOT assay was performed with splenocytes from immunized mice. Splenocytes were harvested aseptically, processed into single cell suspension and plated with 5×105 splenocytes per well in 96-well PVDF plates (EMD Millipore) pre-coated with IFN-γ antibody (R&D Systems) overnight. Splenocytes were then re-stimulated with antigen peptides (2 μg/ml) or controls for 24 hours. Assays were completed using sequential incubations with biotinylated-secondary antibody, streptavidin alkaline phosphatase (Sigma Chemical), and NBT/BCIP substrate (Surmodics). Developed spots were analyzed using an AID iSpot Reader (Autoimmun Diagnostika GmbH, Germany).

Tumor Microenvironment Analysis.

Tumors excised on day 26 after injection were homogenized using pestle motor, and treated with collagenase type IV (1 mg/mL) and DNase I (100 U/mL) in serum-free RPMI for 30 min at 37° C. with gentle shaking. The cell suspension was passed through a cell strainer (70-μm) and washed with FACS buffer twice. Cells were then incubated with CD16/32 blocking antibody for 10 min, and then stained with antibodies for 30 min at room temperature against CD45 (eBiosciene), H-2Kb OVA tetramer-SINNFEKL (MBL International), CD4 (eBioscience), CD8a (BD Bioscience), CD11c (Biolegend), CD11b (Biolegend), F4/80 (Biolegend), CD86 (BD Bioscience), CD3 (Biolegend) and NK1.1 (eBioscience) for flow cytometry analysis.

Statistical Analysis.

For animal studies, mice were randomized to match similar average volume of the primary tumors before the initiation of any treatments. All procedures were performed in a non-blinded fashion. Statistical analysis was performed with Prism 6.0 software (GraphPad Software) by an unpaired student's t-test, one-way or two ANOVA with Bonferroni multiple comparisons post-test. Statistical significance for survival curve was calculated by the log-rank test. Data were approximately normally distributed and variance was similar between the groups. Statistical significance is indicated as *P<0.05, **P<0.01, ***P<0.001, and ****P<0.0001.

Experimental Results

The nanocapsule of the present disclosure is schematically shown in FIGS. 1 and 2. As can be seen from the figures, the nanocapsule system contains a hollow core and an immunostimulatory polysaccharide mimicking an immunogenic bacterial cell wall.

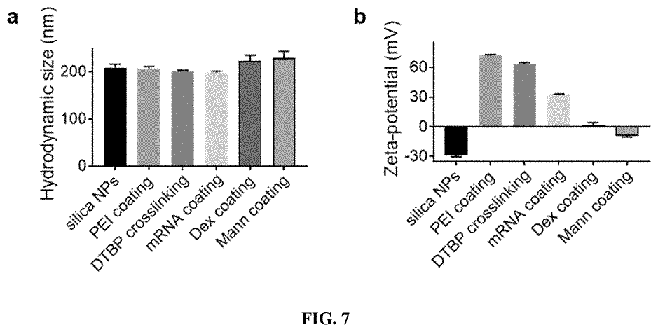

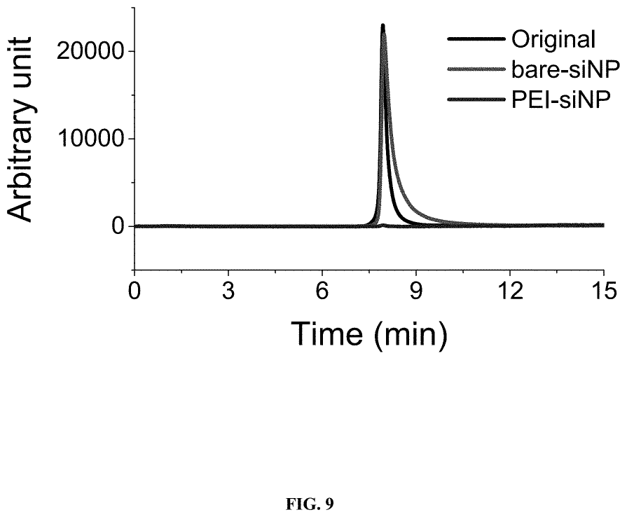

The nanocapsules were constructed on silica nanoparticles (siNPs, 200 nm) employed as a sacrificing template. Specifically, siNPs were first coated with polyethyleneimine (PEI, 25 kDa) that served as a backbone for the capsule structure. PEI was crosslinked using a reduction sensitive-crosslinker, dimethyl 3,3′-Dithiobispropionimidat×2HCl (DTBP), to provide structural robustness and intracellular degradability. mRNA was then loaded into the surface of nanocapsules by charge interaction with cationic PEI (e.g., using layer-by-layer technique), followed by the chemical introduction of oxidized polysaccharides. The siNP core was then removed at the final step to obtain hollow nanocapsules. The spherical nanostructure of nanocapsules was intact after removing the siNP template by ammonium fluoride, and the hollow nanostructure was clearly seen as a collapsed structure in TEM images (FIG. 2b). Compared with the template siNP with 198.7±3.4 nm diameter, the hydrodynamic size of nanocapsules exhibited a slightly increased diameter of about 226±13 nm, and the surface charge was about −10±1.1 mV (FIG. 7 a, b). Interestingly, after mRNA deposition by repeated layer-by-layer coating of mRNA and PEI, nanocapsules maintained their undeformed sherical shape as shown in TEM images, suggesting that LbL deposition of mRNA rendered the capsule structure more rigid (FIG. 2d). The layer of mRNA-loaded nanocapsule was composed of polysaccharide:PEI:mRNA in a ratio of about 40:7:3 by the weight, as indicated by GPC analysis and TNBSA assay (FIG. 2 c and FIG. 9). Nanocapsules were designed to degrade and release mRNA in a reductive intracellular environment by the cleavage of reduction sensitive-crosslinker that maintains PEI backbone. Sugar-nanocapsule was either completely disrupted to debris or swelled to large hollow structures upon DTT treatment as shown by the TEM images (FIG. 2e). More importantly, efficient reduction-sensitive mRNA release from the capsule was confirmed by agarose gel electrophoresis over time upon DTT treatment (FIG. 2f), whereas non-degradable mRNA/PEI complex did not exhibit reduction-sensitive mRNA release.

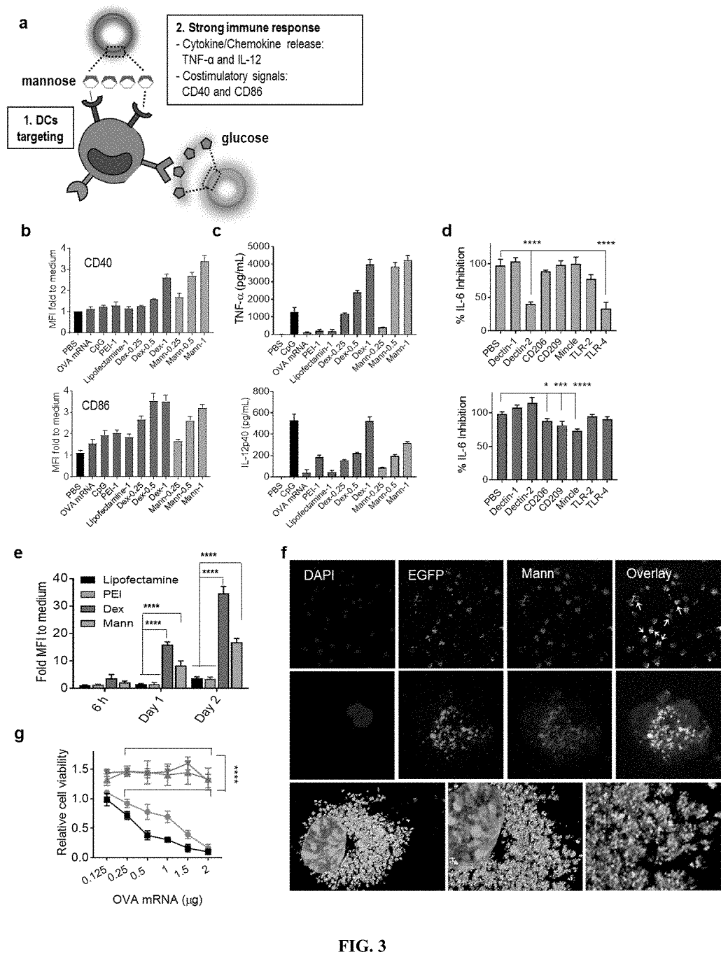

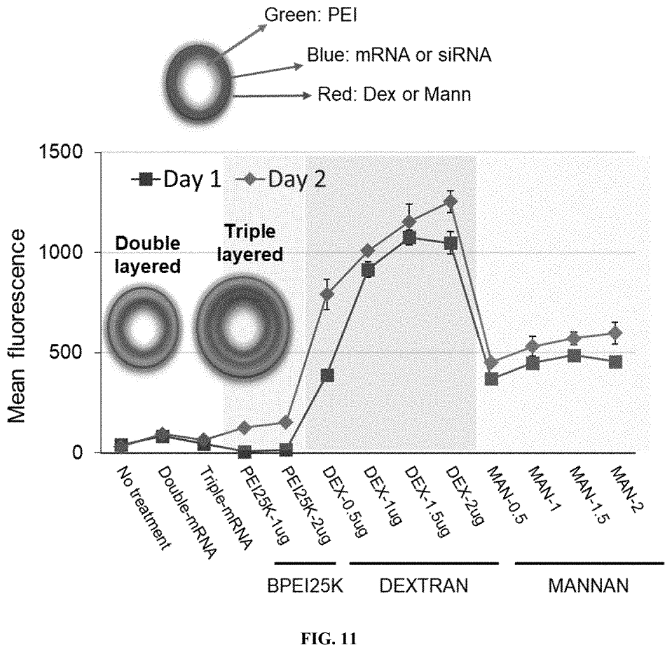

Immunological activities of Sugar-capsules with mRNA encoding a model antigen, ovalbumin (mOVA) were also investigated. Two classes of PAMP-imprinted nanocapsules were constructed, mannose of Mann-capsule or glucose of Dex-capsule, for activation of DCs (FIG. 3a). For comparison, CpG, which is a Toll-like receptor 9 (TLR-9) agonist as a strong PAMP, was employed as a positive control. PEI (cationic polymer) or lipofectamine (cationic liposome) complexed with mOVA were also included as the conventional nucleic acid transfection agents. The upregulation of CD40 and CD86 co-stimulatory markers was confirmed after incubating BMDCs with varying amount of mOVA-nanocapsule ranging from 0.25 to 1 μg of mRNA. Overall, Dex- and Mann-capsules led to dose-dependent upregulation of CD40 and CD86 expression by BMDCs, outperforming other gene transfection agent controls (FIG. 3b and FIG. 11a, b). Similar dose-dependent increase was observed in the secretion of inflammatory cytokines, TNF-α and IL-12p40, with the level comparable to or higher than CpG (FIG. 3c and FIG. 11c). These results clearly demonstrated that Dex- and Mann-capsules interacted with DCs and serve as strong DC activators. To further examine the PAMP-PRR interactions, BMDCs were pre-treated with blocking antibodies against various PRRs and pulsed DCs with Dex- or Mann-capsule, followed by monitoring IL-6 cytokine release (FIG. 2d). The results indicated that several PRRs were crucial in the recognition of Dex- or Mann-capsules by BMDCs. Blocking Dectin-2 and TLR-4 on BMDCs pulsed with Mann-capsules led to significant decrease in IL-6 release, while blocking CD206, CD209, and Mincle on BMDCs pulsed with Dex-capsules significantly degreased IL-6 cytokine secretion. Taken together, these results show that nanocapsules are strong DC activators and that Dex- and Mann-capsules interact with distinct PRRs for DC activation.

Nanocapsules were evaluated as a mRNA delivery platform. Translation of mRNA was evaluated in BMDCs in vitro using mRNA encoding an enhanced green fluorescence protein (EGPF) as a reporter gene. Dex- and Mann-capsules promoted robust expression of EGFP in BMDCs after 1 or 2 days of incubation, whereas PEI and lipofectamine formulations induced only marginal expression (FIG. 3e, FIG. 11). Within 12 hr of incubation with Mann-capsule carrying mRNA, prominent EGFP signals were observed within cytoplasmic region as shown in the confocal microscopy images (FIG. 3f). Dex- and Mann-capsules did not affect the cell viability in all mRNA doses tested, whereas PEI and lipofectamine induced significant dose-dependent cytotoxicity (FIG. 3g). These results demonstrate that nanocapsules serve as a biocompatible and effective mRNA delivery platform to promote mRNA translation in DCs.

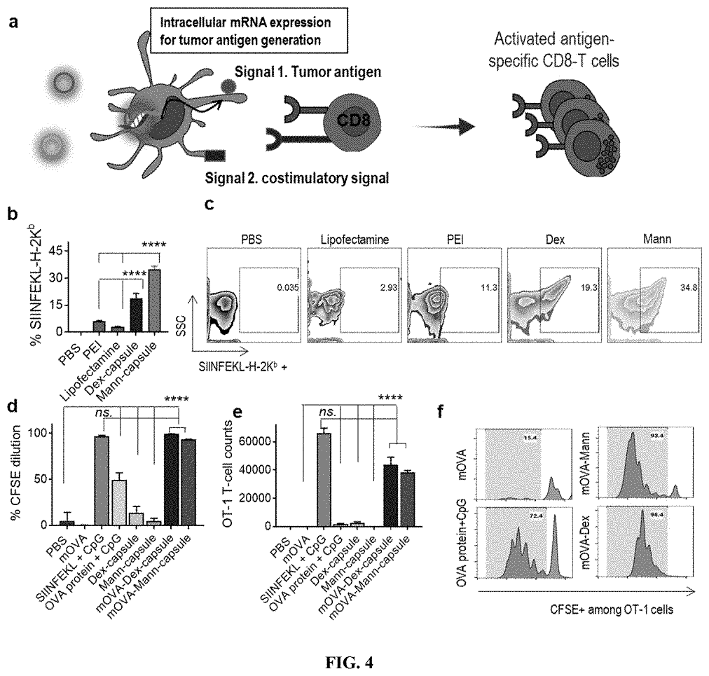

Cancer vaccine aims to amplify antigen-specific T cell responses by promoting DCs to present tumor antigens on MEW molecules (signal 1) and provide costimulatory signals (signal 2) required for the optimal priming of T cells (FIG. 4a). It was determined that nanocapsule-mediated mRNA delivery can result in proper antigen translation and presentation by BMDCs (signal 1). MHC-I presentation of OVA-derived MHC-I minimal epitope, SIINFEKL, was measured after incubating BMDCs with mOVA-nanocapsules. Incubation of BMDCs with Dex- and Mann-capsules for 12 hours led to about 17 and 32% of SIINFEKL-H-2K^{b} positive BMDCs, respectively, which were significantly higher than those induced by PEI and lipofectamine mOVA formulations (FIG. 4 b,c). The extent of antigen presentation was well correlated with the expression efficiency of EGFP mRNA, suggesting that nanocapsules promoted efficient mRNA transfection, translation, and antigen presentation among DCs in vitro.

Efficient antigen presentation by DCs led to robust antigen-specific T-cell proliferation and expansion, as measured by carboxyfluorescein succinimidyl ester (CFSE) dilution assay with SIINFEKL-specific CD8α+ T cells obtained from OT-I transgenic mice (OT-I T cells) (FIG. 3d-f). BMDCs treated with Dex- and Mann-capsule induced almost 100% proliferation of OT-I T cells, which was similar to free SIINFEKL peptide+CpG and significantly higher than free OVA protein+CpG treatment. The number of expanded OT-I T-cells by cross-priming was also significantly higher for nanocapsules than OVA protein+CpG control group. These results demonstrate that nanocapsules can serve as a efficient mRNA-based vaccine platform for eliciting strong antigen-specific T cell responses.

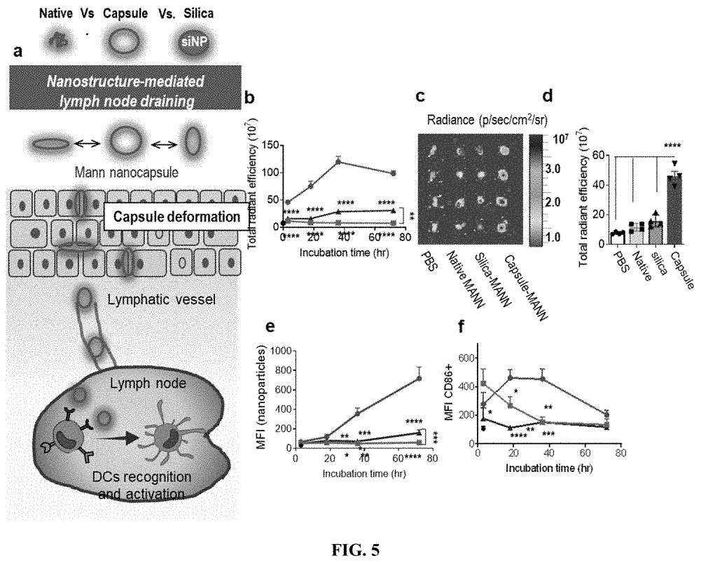

Efficient lymph node drainage is a rate-limiting step for engaging DCs and generating robust T cell responses with in vivo vaccine application. The unique hollow nanostructure of the present nanocapsules promoted lymph node drainage of the nanocapsules through lymphatic vessel by facilitating flexible deformable property upon administration (FIG. 4a). To investigate this, we monitored the kinetics of lymph node drainage by Mann-capsule (capsule-Mann) in comparison with non-particulate, native form of Mann (native-Mann) and non-deformable, rigid Mann-coated silica nanoparticle (silica-Mann) after s.c. tail base injection. The silica-Mann is identical to capsule-Mann in physicochemical properties except for the existence of rigid silica nanoparticle in the core. All formulations were labeled by Cy5.5 fluorophore for IVIS imaging and quantification. Ex vivo fluoresence imaging and quantification demonstrated remarkably higher lymph node accumulation of capsule-Mann compared to native-Mann and silica-Mann formulations throughout 72 hr (FIG. 5 b,c,d). Native-Mann was quickly cleared out from the lymph node after the initial accumulation at 3 hr post-injection presumably due to small molecular size. Majority of silica-Mann, on the other hand, remained at injection site during the period (data not shown) and showed marginal increase in lymph node accumulation at later time points (FIG. 5d). More importantly, within lymph node, capsule-Mann was preferentially taken up by DCs over time and led to the activation, evidenced by the upregulation of CD80 and CD86 markers on DCs (FIG. 5 e,f,g). These results demonstrate that the hollow and deformable nanostructure failicates efficient accumulation and long-term retention of nanocapsule in LNs and promotes activation of DCs, implicating efficient nano-vaccine design.

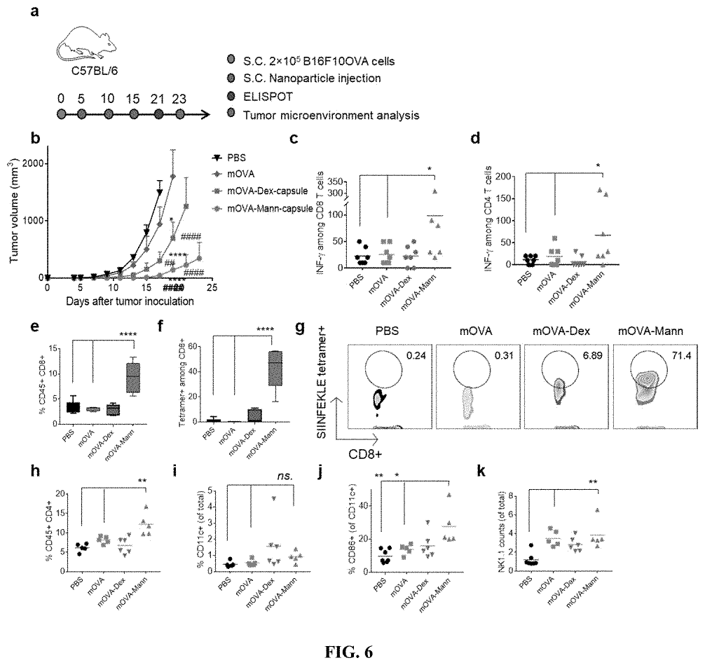

Finally, the anti-tumor therapeutic efficacy of mRNA-nanocapsules was evaluated using B16F10 OVA melanoma model. Mice were inoculated s.c. with B16F100VA cells and when the tumors were palpable on day 5, the animals were vaccinated s.c. at tail base with PBS, mOVA, mOVA-Dex- or mOVA-Mann-capsule (FIG. 6 a). The animals received boost immunizations on days 10 and 15. Mice treated with mOVA-Mann-capsule showed significantly delayed tumor growth while mOVA-Dex-capsule exhibited modest reduction in tumor growth, compared with PBS and mOVA (FIG. 6 b). The degree of tumor suppression by Dex- and Mann-capsule correlated with the MHC I-SIINFEKL presention efficiency as shown in FIG. 4 b, suggesting the involvement of antigen-specific CD8 T cells. To further delineate the key immune cells for the anti-tumor effect, we analyzed splenocytes and tumors on day 21 and 23, respectively. Mann-capsule elicited higher levels of IFN-γ⁺ CD4 and CD8 T cells in spleen (p<0.05 vs. PBS and free mOVA for both CD4 and CD8 T-cell), indicating induction of systemic immune responses (FIG. 6 c,d). In addition, CD8 T-cells infiltrated efficiently into tumors in animals treated with mOVA-Mann-capsule (FIG. 6 e), with a significant portion of the tumor-infiltrating CD8 T cells exhibiting specificity to SIINFEKL peptide (FIG. 6 f,g). Mann-capsule also promoted tumor infiltration of CD4+ T cells, natural cells, and activated DCs, suggesting that Mann-capsule triggered innate and adaptive immune responses for the generation of strong anti-tumor effect (FIG. 6 h-k).

Manipulating Innate Immunity

Innate immune system constitutes an essential arm of host defense against infectious as well as non-infectious diseases including cancers. Non-inflamed tumors lack critical cytokines/chemokines secreted by innate immune cells such as DCs and macrophages, failing to recruit effector CTLs. In inflamed tumors with existing CTLs, many CTLs become largely dysfunctional by the immunosuppressive milieu, created, at least in part, by immunosuppressive innate immune cells such as tumor-associated macrophages (TAMs) and myeloid-derived-suppressor cells (MDSCs). Manipulating innate immunity in tumors is a promising strategy in orchestrating anti-tumor immune responses for unleashing full therapeutic potential of adaptive immunity especially in the context of combination treatments. The local stimulation of innate immunity in tumors triggers in situ vaccination to activate and reinvigorate tumor-specific CTLs through the interplay between innate and adaptive immune networks.

Conventional cancer vaccines employ tumor-associated antigens (TAA) along with immunoadjuvants, which requires to identify and produce antigens specific to individual tumors through time-consuming and technically-demanding processes. In contrast, in situ vaccination is a simple, cost-effective, and practical strategy that can be applied to many cancer types without prior screening of TAA, which can also effectively address the heterogeneity of TME. Many immunoadjuvants have been developed and investigated to activate innate immune cells in local tumors, including Toll-like receptor (TLR) agonists and STING (stimulator of interferon genes) agonists. Despite their effective immune stimulation, their poor physicochemical properties result in unfavorable pharmacokinetic and pharmacodynamics parameters, systemic toxicity, and off-target effects, thereby limiting their clinical use.

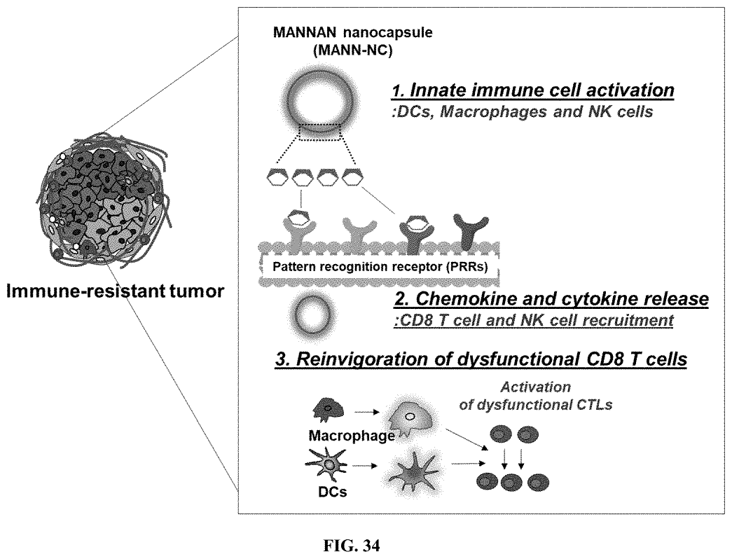

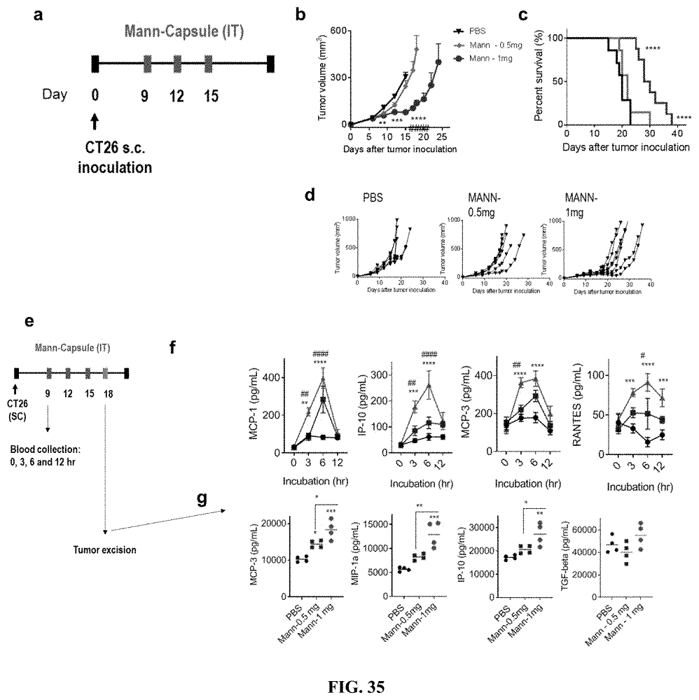

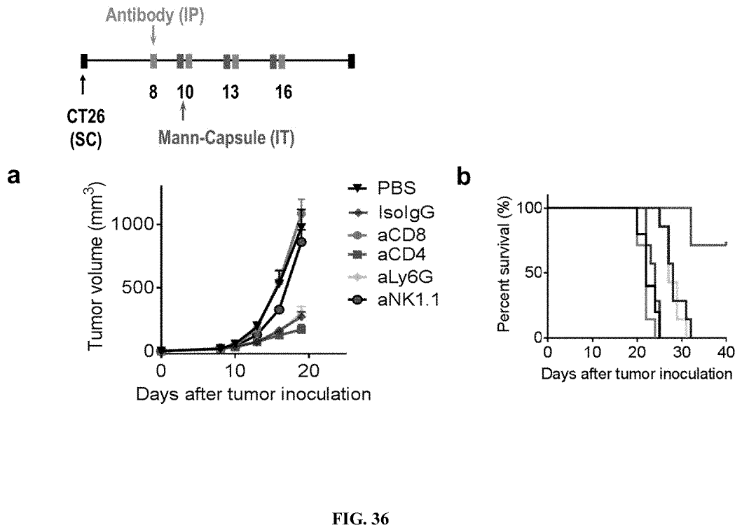

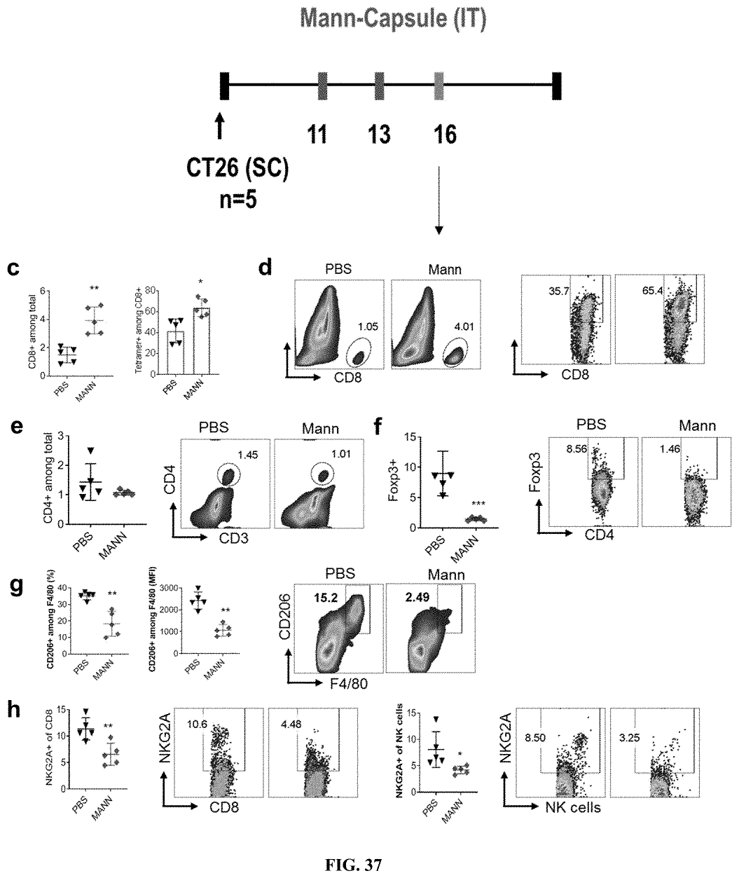

The experimental results described herein show that mannan-based nanocapsules (MANN-NC) act as an immunomodulatory agent that can effectively activate innate immune cells in local tumors and substantially potentiate cancer immunotherapy. Notably, MANN-NC is comprised entirely from mannose repeating unit that can be recognized by PRRs, specifically by mannose receptor (CD206), dectin-1 and 2 and TLRs of innate immune cells. MANN-NC strongly activated DCs and macrophages and promoted the release of pro-inflammatory cytokine/chemokine in vitro, via the recognition by TLR4 and dectin-2. In addition, MANN-NC induced the release of chemoattractants, such as IP-10, MCP-1a, and MCP-3, in locally treated tumors, leading to significant antitumor therapeutic efficacy in CT26 and MC38 murine tumor models through the recruitment and activation of NK cells and T cells and rendering tumor microenvironment significantly less immunosuppressive. See FIGS. 34-37.

### CONCLUSION

The nanocapsules described in the present disclosure advantageously showed strong interaction with antigen-presenting cells (APCs). Specifically, after in vivo administration, nanocapsules within the present claims promoted dramatic elevation of activation markers on dendritic cells (CD86), macrophages (CD80), and natural killer cells (CD107a). The nanocapsules within the present claims showed remarkable therapeutic potential by delivering mRNA as well as peptide-based tumor antigens in murine models of cancer. Mannan-coated nanocapsules were efficiently loaded with mRNA encoding a model antigen ovalbumin (mOVA) (about 100% loading efficiency). Subcutaneous (SC) administration of mannan-based nanocapsules in C57BL/6 mice resulted in efficiently draining to local lymph nodes. C57BL/6 mice bearing B16F10-OVA melanoma tumors were administered SC with mOVA-MANN-NCs, leading to significantly suppressed tumor growth, compared with the soluble mRNA control. Notably, treatments with mOVA-MANN-capsules led to about 3-fold increases in the cell counts for CD4+ and CD8+ T-cells as well as activated DCs (CD86+) and natural killer (NK) cells within tumors. NCs also significantly enhanced the antigen-specific tumor-infiltrating T-cells as evidenced by about 70% OVA tetramer+CD8+ T-cells. In mice with MC3 S colon carcinoma, mannan-based nanocapsules loaded with neo-antigen Adpgk peptide improved induction of NK cells, CD4 and CD8-T cells and exhibited enhanced therapeutic efficacy, compared with soluble adpgk/CpG control. On the other hand, in vivo administration of “blank” dextran-based nanocapsules exhibited anti-tumor efficacy even without the use of any antigens. When dextran-coated nanocapsules were used to load OVA peptide antigen (SIINFEKL), in vivo administration resulted in immune tolerance. C57BL/6 mice treated with SIINFEKL-dextran nanocapsules led to initial expansion of antigen-specific CD8 T cells, but subsequent boost vaccinations decreased CD8 T cells in antigen-specific manner, indicating that dextran-coated nanocapsules mediated T cell deletion or anergy.

### REFERENCES

[1] J. Banchereau, R. M. Steinman, Nature 1998, 392, 245.
[2] T. H. Mogensen, Clinical microbiology reviews 2009, 22, 240.
[3] T. Kawai, S. Akira, Nature immunology 2010, 11, 373; A. Ozinsky, D. M. Underhill, J. D. Fontenot, A. M. Hajjar, K. D. Smith, C. B. Wilson, L. Schroeder, A. Aderem, Proceedings of the National Academy of Sciences of the United States of America 2000, 97, 13766.
[4] K. Fiedler, S. Lazzaro, J. Lutz, S. Rauch, R. Heidenreich, Recent results in cancer research. Fortschritte der Krebsforschung. Progres dans les recherches sur le cancer 2016, 209, 61; C. Grunwitz, L. M. Kranz, in Cancer Vaccines, (Eds: N. Savelyeva, C. Ottensmeier), Springer International Publishing, Cham 2017, 145; N. Pardi, M. J. Hogan, F. W. Porter, D. Weissman, Nature reviews. Drug discovery 2018, 17, 261.
[5] S. Grabbe, H. Haas, M. Diken, L. M. Kranz, P. Langguth, U. Sahin, Nanomedicine (London, England) 2016, 11, 2723.
[6] S. M. G., Nucleic Acid Therapeutics 2018, 28, 158; K. A. Hajj, K. A. Whitehead, Nature Reviews Materials 2017, 2, 17056; B. Li, X. Zhang, Y Dong, Wiley interdisciplinary reviews. Nanomedicine and nanobiotechnology 2018, e1530.
[7] K. Lin, A. M. Kasko, ACS macro letters 2014, 3, 652; A. O. Tzianabos, Clinical microbiology reviews 2000, 13, 523; Q. Zhang, H. S. Overkleeft, G. A. van der Marel, J. D. Codee, Current opinion in chemical biology 2017, 40, 95.
[8] J. Nam, S. Son, J. J. Moon, Cellular and molecular bioengineering 2017, 10, 341.
[9] S. Son, K. Singha, W. J. Kim, Biomaterials 2010, 31, 6344.
[10] B. J. Quah, H. S. Warren, C. R. Parish, Nature protocols 2007, 2, 2049.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A method of making a particle, the method comprising:
i) combining a silica particle with a polyamine to obtain a polyamine-coated silica particle;
ii) combining the polyamine-coated silica particle with a crosslinker to obtain a crosslinked polyamine-coated silica particle;
iii) combining the crosslinked polyamine-coated silica particle with a polysaccharide to obtain a polysaccharide-coated silica particle; and
iv) combining the polysaccharide-coated silica particle with a fluoride source to remove the silica particle to obtain the particle.

2. The method of claim 1, wherein the silica particle is carboxylated.

3. The method of claim 1, wherein the polyamine is polyethyleneimine.

4. The method of claim 1, wherein the crosslinker comprises a —S—S— bridge.

5. The method of claim 4, wherein the crosslinker is 3,3′-dithiobispropionimidate having formula: 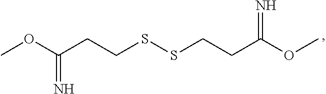
or a salt thereof.

6. The method of claim 1, wherein the polysaccharide comprises at least one aldehyde functional group.

7. The method of claim 1, wherein the polysaccharide comprises a pathogen-associated molecular pattern.

8. The method of claim 1, wherein the polysaccharide is selected from the group consisting of mannan and dextran.

9. The method of claim 1, wherein the fluoride source comprises ammonium fluoride.

10. The method of claim 1, wherein:
the silica particle is carboxylated;
the polyamine is polyethyleneimine;
the crosslinker comprises a —S—S— bridge; and
the polysaccharide is selected from the group consisting of mannan and dextran, and the polysaccharide comprises at least one aldehyde functional group and a pathogen-associated molecular pattern.

11. A particle comprising a shell comprising a crosslinked polyamine layer with a polysaccharide coating, prepared by the method according to claim 1.

12. A particle comprising:
a shell comprising a crosslinked polyamine layer with a polysaccharide coating;
wherein the shell surrounds a hollow core.

13. A pharmaceutical composition comprising a particle according to claim 12, and a pharmaceutically acceptable carrier.

14. A method of modulating an immune response in a subject, the method comprising administering to the subject in need thereof a therapeutically effective amount of a particle of claim 12.

15. The method of claim 14, wherein modulating the immune response comprises modulating a pattern recognition receptor (PRR) on the surface of an antigen-presenting cell (APC), promoting secretion of an inflammatory cytokine, and/or modulating a lymph node drainage.

16. The method of claim 14, wherein the method comprises administering the particle in combination with a vaccine.

17. A method of treating a cancer, the method comprising administering to a subject in need thereof a therapeutically effective amount of a particle of claim 12.

18. The method of claim 17, wherein the method comprises administering the particle in combination with an anti-cancer agent.

19. A method of making a particle, the method comprising:
i) combining a silica particle with a polyamine to obtain a polyamine-coated silica particle;
ii) combining the polyamine-coated silica particle with a crosslinker to obtain a crosslinked polyamine-coated silica particle;
iii) combining the crosslinked polyamine-coated silica particle with a biomolecule to obtain a biomolecule-containing silica particle;
iv) combining the biomolecule-containing silica particle with a polysaccharide to obtain a polysaccharide-coated silica particle; and
iv) combining the polysaccharide-coated silica particle with a fluoride source to remove the silica particle to obtain the particle.

20. The method of claim 19, wherein the biomolecule is selected from the group consisting of a nucleic acid and a therapeutic protein.

21. The method of claim 20, wherein the nucleic acid is a messenger RNA (mRNA) encoding a cancer antigen.

22. The method of claim 20, wherein the therapeutic protein is a cancer antigen or an antibody useful in treating cancer.

23. The method of claim 19, wherein:
the silica particle is carboxylated;
the polyamine is polyethyleneimine;
the crosslinker comprises a —S—S— bridge;
the polysaccharide is selected from the group consisting of mannan and dextran, and the polysaccharide comprises at least one aldehyde functional group and a pathogen-associated molecular pattern; and
the biomolecule is a messenger RNA (mRNA) encoding a cancer antigen.

24. A particle comprising a shell comprising a crosslinked polyamine layer with a polysaccharide coating, and a biomolecule between the crosslinked polyamine layer and the polysaccharide coating, prepared by the method according to claim 19.

25. A particle comprising:
a shell comprising a crosslinked polyamine layer with a polysaccharide coating, and
a biomolecule between the crosslinked layer the polysaccharide coating;
wherein the shell surrounds a hollow core.

26. A pharmaceutical composition comprising a particle according to claim 25, and a pharmaceutically acceptable carrier.

27. A method of modulating an immune response in a subject, the method comprising administering to the subject in need thereof a therapeutically effective amount of a particle of claim 25, wherein the biomolecule is useful in modulating an immune response in the subject.

28. The method of claim 25, wherein modulating the immune response comprises modulating a pattern recognition receptor (PRR) on the surface of an antigen-presenting cell (APC), promoting secretion of an inflammatory cytokine, modulating a lymph node drainage, and/or promoting expression of a disease-specific antigen by the antigen-presenting cell.

29. The method of claim 25, wherein the method comprises administering the particle in combination with an adjuvant.

30. A method of treating a cancer, the method comprising administering to a subject in need thereof a therapeutically effective amount of a particle of claim 25, wherein the biomolecule is useful in treating cancer.
